(19) United States
(12) Patent Application Publication
(10) Pub. No.: US 2026/0306260 A1
(43) Pub. Date: Oct. 8, 2026

(54) DELIVERY SYSTEM FOR PROSTHETIC HEART VALVE

(71) Applicant: THE GLOBAL HEART VALVE INNOVATION CENTER (ISRAEL) LTD, Or Yehuda (IL)

(72) Inventors: Meni IAMBERGER, Kfar Saba (IL); Boaz HARARI, Ganey Tikva (IL); Bar GOLDFARB, Tel Aviv (IL)

(73) Assignee: THE GLOBAL HEART VALVE INNOVATION CENTER (ISRAEL) LTD, Or Yehuda (IL)

(21) Appl. No.: 19/679,509

(22) Filed: May 15, 2026

Related U.S. Application Data
(63) Continuation of application No. PCT/IL2024/051085, filed on Nov. 14, 2024.
(60) Provisional application No. 63/707,415, filed on Oct. 15, 2024.
(60) Provisional application No. 63/599,793, filed on Nov. 16, 2023.

(51) Int. Cl. A61F 2/24

(52) U.S. Cl. CPC A61F 2/2436; A61F 2/2418

(57) ABSTRACT

An implant delivery tool includes proximal and distal capsule portions (51, 53) and an elongate implant holder (40) including a proximal- and distal-capsule-moving-tubes (301, 310). A safety mechanism (300) includes a proximal capsule locker (302), a sleeve (312), and an intermediate tube (314) having a collapsible proximal portion (318) formed of at least two elongate teeth (320) each having a proximal engaging element (322). In a locked state of the safety mechanism (300), a shoulder (308) of the proximal capsule locker (302) is distal to the proximal engaging elements (322) and the sleeve (312) is within the tube (314) such that an outer surface of the sleeve (312) is between elements (322), preventing collapsing of the teeth (320). The proximal engaging elements (322) of the teeth (320) remain engaged with the shoulder (308) and restrict proximal movement of the proximal capsule portion (51). Other embodiments are also described.

### CROSS-REFERENCE TO RELATED APPLICATIONS

The present disclosure is a Continuation Application of PCT Application No.PCT/IL2024/051085, filed on November 14, 2024, which claims the benefit of US Provisional Patent Application 63/599,793 to Iamberger et al., entitled, “Delivery system for prosthetic heart valve,” filed November 16, 2023, and of US Provisional Patent Application 63/707,415 to Iamberger et al., entitled, “Delivery system for prosthetic heart valve,” filed October 15, 2024, which are assigned to the assignee of the present patent application and is incorporated herein by reference.

### FIELD OF THE INVENTION

Some applications of the present invention relate in general to valve replacement. More specifically, some applications of the present invention relate to delivery tools for prosthetic valves for replacement of a cardiac valve.

### BACKGROUND OF THE APPLICATION

Aortic regurgitation (AR), also known as aortic insufficiency (AI), is the leaking of the aortic valve of the heart that causes blood to flow in the reverse direction during ventricular diastole, from the aorta into the left ventricle. As a consequence, the cardiac muscle is forced to work harder than normal. Transcatheter aortic valve replacement (TAVR) is a treatment for aortic valve regurgitation and stenosis.

### SUMMARY OF THE APPLICATION

For some applications, a delivery tool is provided for delivery a prosthetic valve to a heart of a patient. The delivery tool comprises a mechanism for axial and rotational movement of an implant holder relative to the steerable catheter of the delivery tool. The delivery tool also comprises a mechanism for rotationally locking the implant holder relative to the steerable catheter. The delivery tool comprises a handle assembly for controlling such axial and rotational movement. The handle assembly comprises a steerable-catheter-handle portion and elongate-implant-holder-handle portion that is integrated with the steerable handle portion.

The delivery tool also comprises proximal and distal capsule portions for surrounding the implant and a safety mechanism which prevents premature retraction of the capsule portions in order to avoid premature decoupling of the implant from the delivery tool.

The delivery tool comprises steerable catheter assemblies that accomplish independent control of catheter flex magnitude and catheter flex direction and multiple plane steering. A clutch mechanism is integrated into the steerable catheter assembly in order to avoid over-tensioning the pullwire.

There is therefore provided, in accordance with some applications of the invention, a delivery tool for delivering an implant to a heart of a patient, the delivery tool including:

a delivery catheter having a lumen therethrough;

a distal capsule portion configured to house a distal portion of the implant;

a proximal capsule portion configured to house a proximal portion of the implant, the proximal and distal capsule portions being movable with respect to each other;

an elongate implant holder slidable within the lumen of the delivery catheter and reversibly couplable to the implant, the elongate implant holder including:

a proximal-capsule-moving-tube coupled to a proximal end of the proximal capsule portion such that proximal movement of the proximal-capsule-moving-tube moves the proximal capsule portion proximally, and distal movement of the proximal-capsule-moving-tube moves the proximal capsule portion distally;

a distal-capsule-moving-tube coupled to the distal capsule portion such that proximal movement of the distal-capsule-moving-tube moves the distal capsule portion proximally, and distal movement of the distal-capsule-moving-tube moves the distal capsule portion distally; and

a safety mechanism including:

a proximal capsule locker which surrounds and is coupled to the proximal-capsule-moving-tube, the proximal capsule locker being shaped so as to define a midsection protrusion which protrudes into a lumen of the proximal-capsule-moving-tube, the midsection having a midsection-inner-diameter which is narrower than remaining portions of the proximal capsule locker such that the midsection of the proximal capsule locker forms a shoulder;

a locking sleeve coupled to and surrounding the distal-capsule-moving-tube, the locking sleeve having a locking-sleeve-outer diameter, the distal-capsule-moving-tube has a releasing portion proximal to the locking sleeve, the releasing portion having a releasing-portion-outer diameter that is narrower than the locking-sleeve-outer diameter,

an intermediate tube which surrounds the distal-capsule-moving-tube, the intermediate tube having:

an intermediate-tube lumen through which (i) the distal-capsule-moving-tube, and (ii) and the locking sleeve slide,

a distal tubular portion, and

a collapsible proximal portion formed of at least two elongate teeth each having a proximal engaging element,

in a locked state of the safety mechanism:

the shoulder of the proximal capsule locker is disposed distally to the proximal engaging elements of the intermediate tube,

in response to proximal movement of the proximal capsule portion and of the proximal capsule locker by the proximal-capsule-moving-tube, the shoulder of the proximal capsule locker is moved closer to the proximal engaging elements,

in response to continued proximal movement of the proximal capsule portion and of the proximal capsule locker by the proximal-capsule-moving-tube, the shoulder of the proximal capsule locker abuts against the proximal engaging elements, and

the locking sleeve of the distal-capsule-moving-tube is disposed within the lumen of the intermediate tube and in a manner in which an outer surface of the locking sleeve of the distal-capsule-moving-tube is disposed between the proximal engaging elements and prevents collapsing of the at least two elongate teeth such that the proximal engaging elements of the teeth remain engaged with the shoulder of the proximal capsule locker and remain in a state in which the proximal engaging elements restrict further proximal movement of the proximal capsule portion,

following movement of the proximal capsule portion and engaging of the shoulder with the proximal engaging elements, the distal-capsule-moving-tube is configured to be moved axially distally through the intermediate-tube lumen so as to move the distal capsule portion distally and transition the safety mechanism into an unlocked state, and

in response to distal movement of the distal capsule portion by the distal-capsule-moving-tube, the locking sleeve moves distally beyond the proximal engaging elements of the teeth and the releasing portion of the distal-capsule-moving-tube moves within the lumen of the intermediate tube and in between (1) the proximal engaging elements and (2) at least a portion of the at least two elongate teeth in a manner which allows for the collapsing of the at least two elongate teeth such that the proximal engaging elements of the teeth are disengaged from the shoulder, and the further proximal movement of the proximal capsule portion is enabled.

In an application, the delivery catheter includes a steerable catheter.

In an application, the delivery tool includes:

a proximal-capsule-knob configured to control axial movement of the proximal capsule portion; and

a distal-capsule-knob configured to control axial movement of the distal capsule portion.

In an application, the delivery tool includes:

a first steerable-control-knob for controlling lateral steering of a distal portion of the steerable catheter, and

a second steerable-control-knob disposed coaxially with respect to the first steerable-control-knob, the second steerable-control-knob being configured for controlling flexible bending of the distal portion of the steerable catheter.

In an application, the midsection-inner-diameter is 2.0 mm – 2.5 mm.

In an application, the locking-sleeve-outer diameter is 1.5 – 2.0 mm.

In an application, the releasing-portion-outer diameter D3 is 1.0 – 1.5 mm.

In an application, the native heart valve includes a native aortic valve, and the implant includes:

a proximal, downstream skirt which is configured to be expanded downstream of native aortic valve leaflets;

one or more radially-expandable locators configured to abut a downstream surface of the native aortic valve leaflets during the positioning of the implant within the native heart valve; and

a distal, upstream skirt which is configured to be placed upstream of the native aortic valve and sandwich the native aortic valve leaflets between (1) the distal, upstream skirt and (2) the one or more radially-expandable locators and the proximal, downstream skirt.

In an application, the one or more radially-expandable locators are expandable at a position downstream of the native aortic valve leaflets in response to the proximal movement of the proximal capsule portion of the proximal capsule locker by the proximal movement of the proximal-capsule-moving-tube.

In an application, the one or more radially-expandable locators are expandable within a left ventricle of the heart of the patient.

In an application, the distal, upstream skirt is configured to:

be exposed from within the distal capsule portion during the distal movement of the distal-capsule-moving-tube axially through the intermediate-tube lumen, and to the movement of the distal capsule portion distally, and

sandwich the native aortic valve leaflets between (1) the distal, upstream skirt and (2) the one or more radially-expandable locators.

In an application, the proximal capsule portion is further moveable proximally by further movement of the proximal-capsule-moving-tube such that:

a proximal end of the implant is exposed from within the proximal capsule portion,

the proximal, downstream skirt is configured to expand, and

the native aortic valve leaflets are sandwiched between (1) the distal, upstream skirt and (2) the one or more radially-expandable locators and the proximal, downstream skirt.

There is additionally provided, in accordance with some applications of the invention, a method for delivering an implant to a heart of a patient using a delivery tool, the delivery tool including:

a delivery catheter having a lumen therethrough;

a distal capsule portion configured to house a distal portion of the implant;

a proximal capsule portion configured to house a proximal portion of the implant, the proximal and distal capsule portions being movable with respect to each other;

an elongate implant holder slidable within the lumen of the delivery catheter and reversibly couplable to the implant, the elongate implant holder including:

a proximal-capsule-moving-tube coupled to a proximal end of the proximal capsule portion such that proximal movement of the proximal-capsule-moving-tube moves the proximal capsule portion proximally, and distal movement of the proximal-capsule-moving-tube moves the proximal capsule portion distally;

a distal-capsule-moving-tube coupled to the distal capsule portion such that proximal movement of the distal-capsule-moving-tube moves the distal capsule portion proximally, and distal movement of the distal-capsule-moving-tube moves the distal capsule portion distally; and

a safety mechanism including:

a proximal capsule locker which surrounds and is coupled to the proximal-capsule-moving-tube, the proximal capsule locker being shaped so as to define a midsection protrusion which protrudes into a lumen of the proximal-capsule-moving-tube, the midsection having a midsection-inner-diameter which is narrower than remaining portions of the proximal capsule locker such that the midsection of the proximal capsule locker forms a shoulder;

a locking sleeve coupled to and surrounding the distal-capsule-moving-tube, the locking sleeve having a locking-sleeve-outer diameter, the distal-capsule-moving-tube has a releasing portion proximal to the locking sleeve, the releasing portion having a releasing-portion-outer diameter that is narrower than the locking-sleeve-outer diameter,

an intermediate tube which surrounds the distal-capsule-moving-tube, the intermediate tube having:

an intermediate-tube lumen through which (i) the distal-capsule-moving-tube, and (ii) and the locking sleeve slide,

a distal tubular portion, and

a collapsible proximal portion formed of at least two elongate teeth each having a proximal engaging element,

advancing the delivery catheter through vasculature of the patient;

positioning the implant within a native heart valve of the heart, in the step of advancing and the step of positioning the safety mechanism is in a locked state in which the shoulder of the proximal capsule locker is disposed distally to the proximal engaging elements of the intermediate tube;

proximally moving the proximal capsule portion of the proximal capsule locker by proximally moving the proximal-capsule-moving-tube, such that responsively to the proximally moving of the proximal-capsule-moving-tube, moving the shoulder of the proximal capsule locker proximally and closer to the proximal engaging elements;

further proximally moving the proximal capsule portion and the proximal capsule locker by further proximally moving the proximal-capsule-moving-tube, such that in response to the further proximally moving of the proximal-capsule-moving-tube, abutting the shoulder of the proximal capsule locker against the proximal engaging elements, and

in the locked state of the safety mechanism, the locking sleeve of the distal-capsule-moving-tube is disposed within the lumen of the intermediate tube and in a manner in which an outer surface of the locking sleeve of the distal-capsule-moving-tube is disposed between the proximal engaging elements and prevents collapsing of the at least two elongate teeth such that the proximal engaging elements of the teeth remain engaged with the shoulder of the proximal capsule locker and remain in a state in which the proximal engaging elements restrict further proximal movement of the proximal capsule portion,

following the step of further proximally moving the proximal-capsule-moving-tube, transitioning the safety mechanism to an unlocked state by distally moving the distal-capsule-moving-tube axially through the intermediate-tube lumen, and thereby moving the distal capsule portion distally;

responsively to the moving the distal capsule portion distally by the distal-capsule-moving-tube, moving the locking sleeve distally beyond the proximal engaging elements of the teeth, and moving the releasing portion of the distal-capsule-moving-tube within the lumen of the intermediate tube and in between (1) the proximal engaging elements and (2) at least a portion of the at least two elongate teeth in a manner which allows for the collapsing of the at least two elongate teeth such that the proximal engaging elements of the teeth are disengaged from the shoulder; and

yet further proximally moving the proximal capsule portion by yet further proximally moving the proximal-capsule-moving-tube.

In an application, the delivery catheter includes a steerable catheter.

In an application, the delivery tool further includes:

a proximal-capsule-knob configured to control axial movement of the proximal capsule portion; and

a distal-capsule-knob configured to control axial movement of the distal capsule portion.

In an application, the delivery tool further includes:

a first steerable-control-knob for controlling lateral steering of a distal portion of the steerable catheter, and

a second steerable-control-knob disposed coaxially with respect to the first steerable-control-knob, the second steerable-control-knob being configured for controlling flexible bending of the distal portion of the steerable catheter.

In an application, the native heart valve includes a native aortic valve, and the implant includes:

a proximal, downstream skirt which is configured to be expanded downstream of native aortic valve leaflets;

one or more radially-expandable locators configured to abut a downstream surface of the native aortic valve leaflets during the positioning of the implant within the native heart valve; and

a distal, upstream skirt which is configured to be placed upstream of the native aortic valve and sandwich the native aortic valve leaflets between (1) the distal, upstream skirt and (2) the one or more radially-expandable locators and the proximal, downstream skirt.

In an application, proximally moving the proximal capsule portion of the proximal capsule locker by proximally moving the proximal-capsule-moving-tube includes expanding the one or more radially-expandable locators at a position downstream of the native aortic valve leaflets.

In an application, the method further includes advancing the proximal capsule portion within a left ventricle prior to positioning the implant within the native heart valve.

In an application, proximally moving the proximal capsule portion of the proximal capsule locker by proximally moving the proximal-capsule-moving-tube includes expanding the one or more radially-expandable locators within the left ventricle, and the method further includes:

proximally withdrawing the one or more radially-expandable locators to a position downstream of the native aortic valve leaflets; and

distally advancing the implant such that the one or more radially-expandable locators abut the downstream surface of the native aortic valve leaflets.

In an application, proximally moving the proximal capsule portion of the proximal capsule locker by proximally moving the proximal-capsule-moving-tube includes exposing the one or more radially-expandable locators from within the proximal capsule portion and expanding the one or more radially-expandable locators, and the method further includes distally advancing the implant such that the one or more radially-expandable locators abut the downstream surface of the native aortic valve leaflets.

In an application, distally moving the distal-capsule-moving-tube axially through the intermediate-tube lumen, and thereby moving the distal capsule portion distally, includes exposing the distal, upstream skirt from within the distal capsule portion and sandwiching the native aortic valve leaflets between (1) the distal, upstream skirt and (2) the one or more radially-expandable locators.

In an application, yet further proximally moving the proximal capsule portion by yet further proximally moving the proximal-capsule-moving-tube includes exposing a proximal end of the implant from within the proximal capsule portion and sandwiching the native aortic valve leaflets between (1) the distal, upstream skirt and (2) the one or more radially-expandable locators and the proximal, downstream skirt.

In an application, exposing the proximal end of the implant from within the proximal capsule portion includes disengaging the implant from the delivery tool.

There is further provided, in accordance with some applications of the invention, a delivery tool for delivering an implant to a heart of a patient, the delivery tool including:

a steerable catheter having a proximal end, a distal end, and a lumen extending between the proximal end and the distal end of the steerable catheter;

an elongate implant holder reversibly couplable to the implant, the elongate implant holder having a proximal end and a distal end, the elongate implant holder being disposed within the lumen of the steerable catheter and being slidable with respect to the steerable catheter in order to advance the implant toward and away from the distal end of the steerable catheter; and

a handle assembly including:

a steerable-catheter-handle portion coupled to the proximal end of the steerable catheter, the steerable-catheter-handle portion having a lumen therethrough and shaped so as to define a proximal receiving space; and

an elongate-implant-holder-handle portion:

coupled to the proximal end of the elongate implant holder, and

integrated with the steerable-catheter-handle portion,

the steerable-catheter-handle portion and the elongate-implant-holder-handle portion are coaxially aligned along a central longitudinal axis of the handle assembly,

a proximal portion of the elongate implant holder is slidable within the lumen of the steerable-catheter-handle portion,

a distal portion of the elongate-implant-holder-handle portion is slidable within the proximal receiving space of the steerable-catheter-handle portion, and

the elongate-implant-holder-handle portion is movable proximally and distally with respect to the steerable-catheter-handle portion along the central longitudinal axis of the handle assembly in a manner which:

in a fully-elongated state of the handle assembly, in which the elongate-implant-holder-handle portion is in a proximal position with respect to the steerable-catheter-handle portion, a proximal end of the elongate-implant-holder-handle portion is disposed farthest from a proximal end of the steerable-catheter-handle portion such that a proximal end of the implant is disposed closest to the distal end of the steerable catheter; and

in a fully-shortened state of the handle assembly, in which the elongate-implant-holder-handle portion has been moved distally with respect to the steerable-catheter-handle portion, the elongate implant holder is advanced distally with respect to the steerable catheter such that the proximal end of the elongate-implant-holder-handle portion is disposed closer to the proximal end of the steerable-catheter-handle portion than in the fully-elongated state, such that the proximal end of the implant is disposed further from the distal end of the steerable catheter than in the fully-elongated state, and a gap is created between the distal end of the steerable catheter and the implant.

In an application, the proximal receiving space has a length of 45-55mm.

In an application, the steerable-catheter-handle portion includes a knob, and the elongate-implant-holder-handle portion is movable by axial movement of the elongate-implant-holder-handle portion while the knob of the steerable-catheter-handle portion is rotated.

In an application, the steerable-catheter-handle portion includes:

a first steerable-control-knob for controlling lateral steering of a distal portion of the steerable catheter, and

a second steerable-control-knob disposed coaxially with respect to the first steerable-control-knob, the second steerable-control-knob being configured for controlling flexible bending of the distal portion of the steerable catheter.

In an application, the gap is 0-6 cm.

In an application, the elongate implant holder includes a hydrophilic coating.

In an application, the elongate-implant-holder-handle portion includes a distal adapter portion which includes a rotational adapter which facilitates rotation of (1) the elongate-implant-holder-handle portion, the elongate implant holder, and the implant with respect to (2) the steerable-catheter-handle portion and to the steerable catheter.

In an application:

the rotational adapter includes an adapter tube shaped so as to define a first coupling, and

the steerable-catheter-handle portion includes a surrounding-ring shaped so as to define a second coupling configured matingly coupled with the first coupling of the adapter tube.

In an application, the first coupling includes at least one female coupling and the second coupling includes at least one corresponding male coupling.

In an application:

the surrounding-ring is shaped so as to define the second coupling at an inner surface of the surrounding-ring and a plurality of teeth arranged circumferentially around an outer surface of the surrounding-ring, and

the surrounding-ring is shaped so as to define a plurality of spaces alternatingly disposed with respect to the plurality of teeth.

In an application, the steerable-catheter-handle portion further includes a rotational locking mechanism including:

a flexible ring surrounding the surrounding-ring, the flexible ring including a spring-loaded engagement mechanism including:

a spring; and

at least one engagement tooth coupled to the spring; and

a button coupling to the flexible ring,

when the button is not pressed, in a relaxed state of the flexible ring, the spring is relaxed in a manner in which the at least one engagement tooth is disposed within at least one of the spaces that are alternatingly disposed with respect to the plurality of teeth of the surrounding-ring in a manner in which the elongate-implant-holder-handle portion is rotationally locked with respect to the steerable-catheter-handle portion, and

when the button is pressed, the button applies a pushing force to the flexible ring to expand a diameter of the flexible ring in a manner in which the flexible ring applies a pushing force to the spring in order to disengage the at least one engagement tooth from within the at least one of the spaces of the surrounding-ring in a manner in which the elongate-implant-holder-handle portion is able to rotate with respect to the steerable-catheter-handle portion.

In an application, the delivery tool further includes at least one capsule for housing the implant in a radially compressed state during delivery, and the elongate-implant-holder-handle portion includes one or more knobs for controlling the unsheathing of the implant from within the capsule.

In an application, the at least one capsule includes:

a distal capsule portion configured to house a distal portion of the implant, and

a proximal capsule portion configured to house a proximal portion of the implant, and

the one or more knobs includes a proximal-capsule-knob for controlling movement of the proximal capsule portion, and a distal-capsule-knob for controlling movement of the distal capsule portion.

In an application, the delivery tool further includes at least one capsule for housing the implant in a radially compressed state during delivery, in the fully-elongated state of the handle assembly, the distal end of the steerable catheter is disposed closest to a proximal end of the capsule, and in the fully-shortened state of the handle assembly, the distal end of the steerable catheter is disposed farther from the proximal end of the capsule.

In an application, the at least one capsule includes:

a distal capsule portion configured to house a distal portion of the implant, and

a proximal capsule portion configured to house a proximal portion of the implant,

the proximal and distal capsule portions are movable with respect to each other.

A kit includes the delivery tool and the kit includes packaging housing the delivery tool, when the delivery tool is disposed within the packaging, the distal portion of the elongate-implant-holder-handle portion is disposed within the at least a proximal portion of the proximal receiving space of the steerable-catheter-handle portion.

In an application, the elongate-implant-holder-handle portion is shaped so as to define a threaded portion having a first thread, and the steerable-catheter-handle portion is shaped so as to define a second thread matingly coupled with the first thread when the delivery tool is disposed within the packaging.

There is yet further provided, in accordance with some applications of the invention apparatus, including a delivery tool including:

a steerable catheter;

a pullwire coupled to the steerable catheter and configured to control steering of the steerable catheter;

a handle assembly coupled to the steerable catheter, the handle assembly including:

a steerable-control-knob coupled to the steerable catheter and operably coupled to the pullwire such that rotation of the steerable-control-knob applies tension to the pullwire;

a flexing-control-screw;

a gear coupled to the flexing-control screw;

a spring integrated within the steerable-control-knob circumferentially around the gear; and

a pin coupled to the spring,

the pin:

is configured to engage spaces between teeth of the gear when torque is applied to the pullwire under a threshold, and

is configured to be automatically disengaged from the spaces between the teeth of the gear when torque is applied to the pullwire above the threshold.

There is also provided, apparatus, including a delivery tool including:

a steerable catheter;

a first steerable assembly configured to control flexible bending of a distal portion of the steerable catheter, the first steerable assembly including a flexing-control-screw-housing and a flexing-control-screw disposed within the flexing-control-screw-housing; and

a second steerable assembly configured to control lateral steering of the distal portion of the steerable catheter the second steerable assembly including:

a first screw; and

a second screw,

the first and second screws are configured to move axially in opposite directions,

the first and second screws are disposed radially externally to the flexing-control-screw-housing, and

the first and second screws and the flexing-control-screw-housing are configured to axially overlap at a given time during use of the delivery tool.

There is additionally provided, in accordance with some applications of the invention a delivery tool for delivering an implant to a heart of a patient, the delivery tool including:

a steerable catheter having a proximal end, a distal end, and a lumen extending between the proximal end and the distal end of the steerable catheter;

an elongate implant holder reversibly couplable to the implant, the elongate implant holder having a proximal end and a distal end, the elongate implant holder being disposed within the lumen of the steerable catheter and being slidable with respect to the steerable catheter in order to advance the implant toward and away from the distal end of the steerable catheter;

a handle assembly including:

a steerable-catheter-handle portion coupled to the proximal end of the steerable catheter, the steerable-catheter-handle portion having a lumen therethrough and shaped so as to define a proximal receiving space;

an elongate-implant-holder-handle portion:

coupled to the proximal end of the elongate implant holder, and

integrated with the steerable-catheter-handle portion;

a first steerable assembly disposed within the steerable-catheter-handle portion and configured to control flexible bending of a distal portion of the steerable catheter, the first steerable assembly including a flexing-control-screw-housing and a flexing-control-screw disposed within the flexing-control-screw-housing; and

a second steerable assembly disposed within the elongate-implant-holder-handle portion and configured to control lateral steering of the flexible tube of the elongate implant holder, the second steerable assembly including:

a distal adapter portion which includes a rotational adapter which facilitates rotation of (1) the elongate-implant-holder-handle portion, the elongate implant holder, and the implant with respect to (2) the steerable-catheter-handle portion and to the steerable catheter, the rotational adapter including an adapter tube shaped so as to define a first coupling;

a surrounding-ring shaped so as to define a second coupling configured matingly coupled with the first coupling of the adapter tube; and

a flexible ring surrounding the surrounding-ring, the flexible ring including a spring-loaded engagement mechanism including:

a spring; and

at least one engagement tooth coupled to the spring; and

a button coupling to the flexible ring,

when the button is not pressed, in a relaxed state of the flexible ring, the spring is relaxed in a manner in which the at least one engagement tooth is disposed within at least one of the spaces that are alternatingly disposed with respect to the plurality of teeth of the surrounding-ring in a manner in which the elongate-implant-holder-handle portion is rotationally locked with respect to the steerable-catheter-handle portion, and

when the button is pressed, the button applies a pushing force to the flexible ring to expand a diameter of the flexible ring in a manner in which the flexible ring applies a pushing force to the spring in order to disengage the at least one engagement tooth from within the at least one of the spaces of the surrounding-ring in a manner in which the elongate-implant-holder-handle portion is able to rotate with respect to the steerable-catheter-handle portion in order to laterally steer the distal portion of the elongate implant holder.

In an application:

the steerable-catheter-handle portion and the elongate-implant-holder-handle portion are coaxially aligned along a central longitudinal axis of the handle assembly,

a proximal portion of the elongate implant holder is slidable within the lumen of the steerable-catheter-handle portion, and

a distal portion of the elongate-implant-holder-handle portion is slidable within the proximal receiving space of the steerable-catheter-handle portion.

In an application:

the elongate-implant-holder-handle portion is movable proximally and distally with respect to the steerable-catheter-handle portion along the central longitudinal axis of the handle assembly in a manner which:

in a fully-elongated state of the handle assembly, in which the elongate-implant-holder-handle portion is in a proximal position with respect to the steerable-catheter-handle portion, a proximal end of the elongate-implant-holder-handle portion is disposed farthest from a proximal end of the steerable-catheter-handle portion such that a proximal end of the implant is disposed closest to the distal end of the steerable catheter, and

in a fully-shortened state of the handle assembly, in which the elongate-implant-holder-handle portion has been moved distally with respect to the steerable-catheter-handle portion, the elongate implant holder is advanced distally with respect to the steerable catheter such that the proximal end of the elongate-implant-holder-handle portion is disposed closer to the proximal end of the steerable-catheter-handle portion than in the fully-elongated state, such that the proximal end of the implant is disposed further from the distal end of the steerable catheter than in the fully-elongated state, and a gap is created between the distal end of the steerable catheter and the implant.

There is also provided, in accordance with some applications of the invention, a method for delivering an implant to a heart of a patient, the method including:

introducing into a body of the patient, a delivery tool including:

a steerable catheter having a proximal end, a distal end, and a lumen extending between the proximal end and the distal end of the steerable catheter;

an elongate implant holder reversibly couplable to the implant, the elongate implant holder having a proximal end and a distal end, the elongate implant holder being disposed within the lumen of the steerable catheter and being slidable with respect to the steerable catheter in order to advance the implant toward and away from the distal end of the steerable catheter;

a handle assembly including:

a steerable-catheter-handle portion coupled to the proximal end of the steerable catheter, the steerable-catheter-handle portion having a lumen therethrough and shaped so as to define a proximal receiving space;

an elongate-implant-holder-handle portion:

coupled to the proximal end of the elongate implant holder, and

integrated with the steerable-catheter-handle portion;

a first steerable assembly disposed within the steerable-catheter-handle portion and configured to control flexible bending of a distal portion of the steerable catheter, the first steerable assembly including a flexing-control-screw-housing and a flexing-control-screw disposed within the flexing-control-screw-housing; and

a second steerable assembly disposed within the elongate-implant-holder-handle portion and configured to control lateral steering of the flexible tube of the elongate implant holder, the second steerable assembly including:

a distal adapter portion which includes a rotational adapter which facilitates rotation of (1) the elongate-implant-holder-handle portion, the elongate implant holder, and the implant with respect to (2) the steerable-catheter-handle portion and to the steerable catheter, the rotational adapter including an adapter tube shaped so as to define a first coupling;

a surrounding-ring shaped so as to define a second coupling configured matingly coupled with the first coupling of the adapter tube; and

a flexible ring surrounding the surrounding-ring, the flexible ring including a spring-loaded engagement mechanism including:

a spring; and

at least one engagement tooth coupled to the spring; and

a button coupling to the flexible ring,

when the button is not pressed, in a relaxed state of the flexible ring, the spring is relaxed in a manner in which the at least one engagement tooth is disposed within at least one of the spaces that are alternatingly disposed with respect to the plurality of teeth of the surrounding-ring in a manner in which the elongate-implant-holder-handle portion is rotationally locked with respect to the steerable-catheter-handle portion, and

when the button is pressed, the button applies a pushing force to the flexible ring to expand a diameter of the flexible ring in a manner in which the flexible ring applies a pushing force to the spring in order to disengage the at least one engagement tooth from within the at least one of the spaces of the surrounding-ring in a manner in which the elongate-implant-holder-handle portion is able to rotate with respect to the steerable-catheter-handle portion in order to laterally steer the distal portion of the elongate implant holder;

pushing the button; and

steering the distal portion of the elongate implant holder by rotating the elongate-implant-holder-handle portion.

In an application:

the steerable-catheter-handle portion and the elongate-implant-holder-handle portion are coaxially aligned along a central longitudinal axis of the handle assembly,

a proximal portion of the elongate implant holder is slidable within the lumen of the steerable-catheter-handle portion,

a distal portion of the elongate-implant-holder-handle portion is slidable within the proximal receiving space of the steerable-catheter-handle portion, and

the method further includes sliding the distal portion of the elongate-implant-holder-handle portion within the proximal receiving space of the steerable-catheter-handle portion.

In an application:

the elongate-implant-holder-handle portion is movable proximally and distally with respect to the steerable-catheter-handle portion along the central longitudinal axis of the handle assembly in a manner which:

in a fully-elongated state of the handle assembly, in which the elongate-implant-holder-handle portion is in a proximal position with respect to the steerable-catheter-handle portion, a proximal end of the elongate-implant-holder-handle portion is disposed farthest from a proximal end of the steerable-catheter-handle portion such that a proximal end of the implant is disposed closest to the distal end of the steerable catheter,

in a fully-shortened state of the handle assembly, in which the elongate-implant-holder-handle portion has been moved distally with respect to the steerable-catheter-handle portion, the elongate implant holder is advanced distally with respect to the steerable catheter such that the proximal end of the elongate-implant-holder-handle portion is disposed closer to the proximal end of the steerable-catheter-handle portion than in the fully-elongated state, such that the proximal end of the implant is disposed further from the distal end of the steerable catheter than in the fully-elongated state, and a gap is created between the distal end of the steerable catheter and the implant, and

the method further includes distally moving the elongate-implant-holder-handle portion with respect to the steerable-catheter-handle portion such that the handle assembly assumes the fully-shortened state, and by the distally moving, creating a gap between the distal end of the steerable catheter and the implant.

### BRIEF DESCRIPTION OF THE DRAWINGS

FIGS. 1-2 are schematic illustrations showing a delivery tool, in accordance with some applications of the present invention;

FIGS. 3A-3B are schematic illustrations of an integrated handle portion of the delivery tool that controls axial movement of respective elongate tubes of the delivery tool, in accordance with some applications of the present invention;

FIGS. 4A-4D are schematic illustrations of the integrated handle portion of the delivery tool that controls rotational movement of respective elongate tubes of the delivery tool, in accordance with some applications of the present invention;

FIGS. 5A-5D are schematic illustrations of a safety mechanism of the delivery tool, which controls unsheathing of the implant delivered by the delivery tool, in accordance with some applications of the invention;

FIGS. 6A-6D are schematic illustrations of steerable assemblies which control steering and flexing of the tip of the steerable catheter along multiple planes, in accordance with some applications of the invention; and

FIG. 7 is a schematic illustration of a clutch mechanism which prevents over torquing of the pullwire that controls the flexing of the distal tip of the steerable catheter, in accordance with some applications of the invention.

The present invention will be more fully understood from the following detailed description of applications thereof, taken together with the drawings, in which:

### DETAILED DESCRIPTION OF EMBODIMENTS

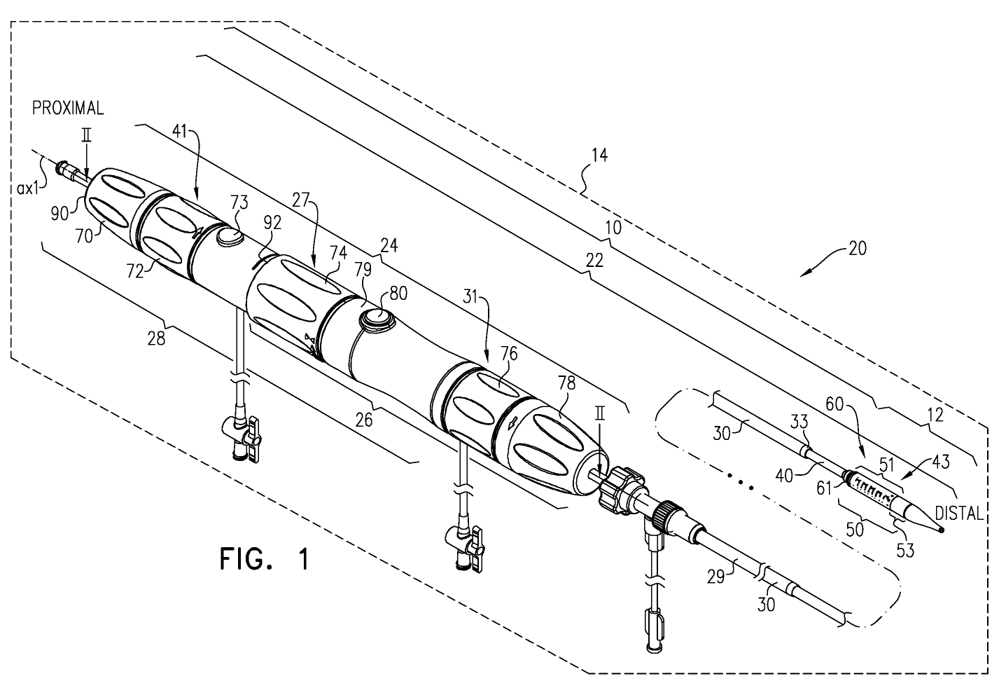

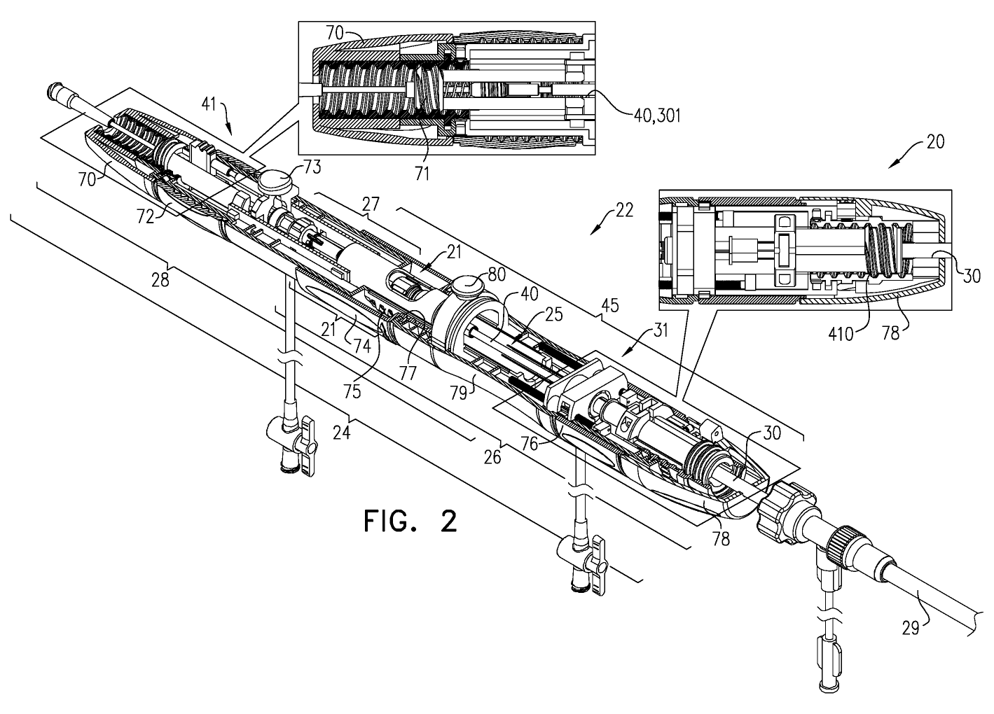

Reference is now made to FIGS. 1, 2, and 3A-B, which are schematic illustrations of a system 20 comprising a delivery tool 22 which has an extracorporeal, integrated handle assembly 24, in accordance with some applications of the present invention. As shown in FIGS. 1 and 2, delivery tool 22 is a multi-catheter transluminal (e.g., transfemoral) delivery tool, comprising three primary components: a catheter system 10, an implantation instrument 12, and extracorporeal, integrated handle assembly 24. Delivery tool 22 is used for delivering an implant 60 to a heart of a patient. Typically, implant 60 comprises a prosthetic heart valve for replacement of a native heart valve, e.g., an aortic valve, or any other heart valve of the patient.

Throughout this application, including the specification and the claims, unless stated otherwise, "upstream" and "downstream," e.g., with respect to the ends of the prosthetic valve of implant 60, are defined with respect to the longitudinal axis of implant 60, by the orientation and functioning of the prosthetic leaflets, which facilitate one-way upstream-to-downstream fluid flow through the lumen. Throughout this application, including the specification and the claims, unless stated otherwise, "proximal" and "distal," e.g., with respect to the direction of delivery. That is, proximal means closer to the point of access into the patient, and distal mean further from the point of access.

Delivery tool 22 comprises a steerable catheter 30 having a proximal end 31, a distal end 33, and a lumen extending between proximal end 31 and distal end 33. As is described hereinbelow, steerable catheter 30 comprises a flexible tube, e.g., a hypotube or comprises a soft polyether block amide (e.g., PEBAX (TM)), or any other suitable flexible material, that has multiple plane steering capabilities, e.g., biplane steering capabilities. For example, steerable catheter 30 may be laterally steered (e.g., anterior-posterior and/or medial-lateral steering) and may be flexed proximally and distally (e.g., superior-inferior steering). Steerable catheter 30 is slidable within an elongate transluminal (e.g., transfemoral) sheath 29. For some applications, steerable catheter 30 comprises the hypotube or comprises a soft polyether block amide at the distal end portion.

Delivery tool 22 comprises an elongate implant holder 40 (e.g., a flexible tube) reversibly couplable to implant 60. For some applications, elongate implant holder 40 comprises a hydrophilic coating. Typically, implant holder 40 is reversibly couplable to implant 60 by being coupled to a proximal capsule portion 51 of at least one capsule 50 that houses implant 60. Elongate implant holder 40 has a proximal end 41 and a distal end 43. Elongate implant holder 40 is disposed and slidable within the lumen of steerable catheter 30 and is slidable with respect to steerable catheter 30 in order to advance implant 60 toward and away from distal end 33 of steerable catheter 30.

Handle assembly 24 comprises:

a steerable-catheter-handle portion 26 coupled to proximal end 31 of steerable catheter 30. Steerable-catheter-handle portion 26 has a lumen 25 therethrough and is shaped so as to define a proximal receiving space 21, and

an elongate-implant-holder-handle portion 28 coupled to proximal end 41 of elongate implant holder 40. Elongate-implant-holder-handle portion 28 is disposed proximally to and is integrated with and mated to steerable-catheter-handle portion 26.

Steerable-catheter-handle portion 26 and elongate-implant-holder-handle portion 28 are coaxially aligned along a central longitudinal axis ax1 of handle assembly 24. As shown in FIG. 2, a proximal portion 45 of elongate implant holder 40 is axially movable within lumen 25 of steerable-catheter-handle portion 26. A distal portion 27 of elongate-implant-holder-handle portion 28 is axially movable within proximal receiving space 21 of steerable-catheter-handle portion 26.

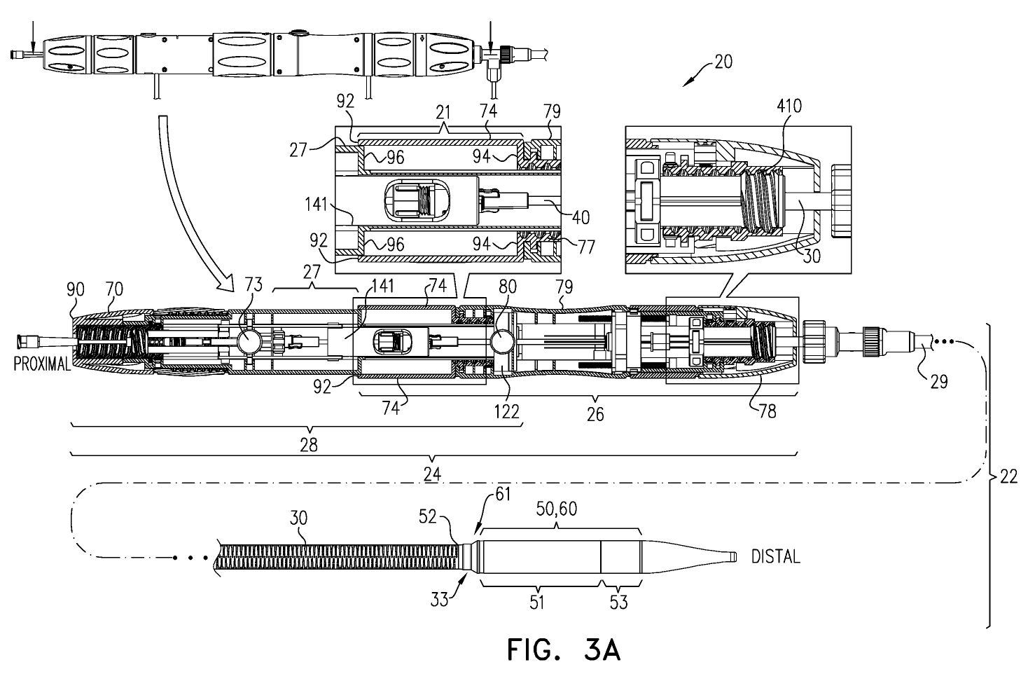

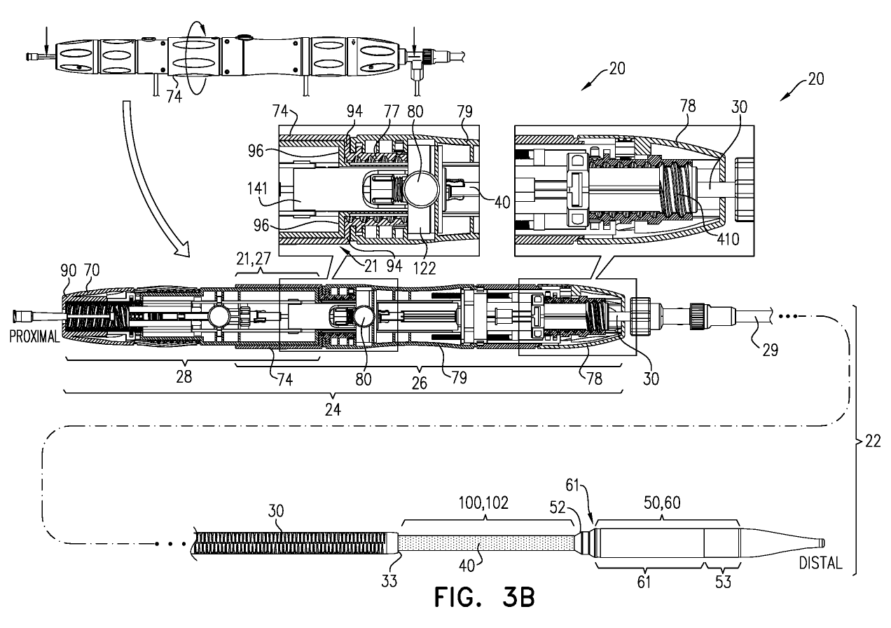

As is shown in FIGS. 3A-B, elongate-implant-holder-handle portion 28 is movable axially proximally and distally with respect to steerable-catheter-handle portion 26 along central longitudinal axis ax1 of handle assembly 24 in a manner which:

in a fully-elongated state (FIG. 3A) of handle assembly 24, elongate-implant-holder-handle portion 28 is in a proximal position with respect to steerable-catheter-handle portion 26 such that a proximal end 90 of elongate-implant-holder-handle portion 28 is disposed farthest from a proximal end 92 of steerable-catheter-handle portion 26 such that a proximal end 61 of implant 60 and a proximal end 52 of proximal capsule portion 51 are disposed closest to distal end 33 of steerable catheter 30; and

in a fully-shortened state (FIG. 3B) of handle assembly 24, elongate-implant-holder-handle portion 28 has been distally moved with respect to steerable-catheter-handle portion 26 such that elongate implant holder 40 is advanced distally with respect to steerable catheter 30 such that proximal end 90 of elongate-implant-holder-handle portion 28 is disposed closer to proximal end 92 of steerable-catheter-handle portion 26 than in the fully-elongated state, such that proximal end 61 of implant 60 and proximal end 52 of proximal capsule portion 51 are disposed further from distal end 33 of steerable catheter 30 than in the fully-elongated state, and a gap 100 is created between distal end 33 of steerable catheter 30 and implant 60, i.e., proximal end 61 of implant 60 and proximal end 52 of proximal capsule 51.

Steerable-catheter-handle portion 26 comprises a gripper, e.g., an axial-movement knob 74. Elongate-implant-holder-handle portion 28 is movable by axial movement of elongate-implant-holder-handle portion 28 while knob 74 of steerable-catheter-handle portion 26 is rotated. Steerable-catheter-handle portion 26 and elongate-implant-holder-handle portion 28 are integrated by being coupled together via respective threading between corresponding portions of steerable-catheter-handle portion 26 and elongate-implant-holder-handle portion 28, as shown in FIG. 2. Elongate-implant-holder-handle portion 28 has a threaded portion which is shaped to define a first thread 75 matingly coupled with a corresponding second thread 77 of the steerable-catheter-handle portion 26, e.g., the threading of axial-movement knob 74. The mating of handle portions 26 and 28 creates an integrated handle assembly. In addition, the mating of handle portions 26 and 28 enables axial movement of elongate-implant-holder-handle portion 28 with respect to steerable-catheter-handle portion 26. Typically, knob 74 is held by and rotatable with respect to a structural housing 79. Thread 77 of the steerable-catheter-handle portion 26 (e.g., the threading of axial-movement knob 74) is surrounded by structural housing 79, as shown in FIG. 2.

Elongate-implant-holder-handle portion 28 is moved axially with respect to steerable-catheter-handle portion 26 such that distal portion 27 of elongate-implant-holder-handle portion 28 is slidable within proximal receiving space 21 of steerable-catheter-handle portion 26 (FIGS. 3A-B). Steerable-catheter-handle portion 26 is shaped so as to define an opening at proximal end 92. The opening opens to a cylindrical wall and a receiving-space-distal wall 94 which define proximal receiving space 21. Distal portion 27 is shaped so as to define a distal wall 96. As shown in FIG. 3B, distal portion 27 of elongate-implant-holder-handle portion 28 is slidable within proximal receiving space 21 of steerable-catheter-handle portion 26 until distal wall 96 of distal portion 27 abuts against receiving-space-distal wall 94 of proximal receiving space 21.

Elongate implant holder 40 is coupled to a pusher 141 at a proximal portion thereof. During movement of handle portion 28 with respect to handle portion 26, pusher 141 moves along with movement of handle portion 28 in order to move, in turn, elongate holder 40.

Typically, proximal receiving space 21 has a length of 45-55 mm, e.g., 47 mm.

As distal portion 27 of elongate-implant-holder-handle portion 28 slides distally within proximal receiving space 21 of steerable-catheter-handle portion 26, elongate implant holder 40 is advanced distally with respect to steerable catheter 30 such that a distal portion of elongate implant holder 40 is exposed from within steerable catheter 30 which pushes implant 60 and capsule 50 distally. During the advancing distally of the distal portion of elongate implant holder 40 beyond distal end 33 of steerable catheter, proximal end 61 of implant 60 and proximal end 52 of proximal capsule 51 are disposed further from distal end 33 of steerable catheter 30 than in the fully-elongated state of handle assembly 24 shown in FIG. 3A, and gap 100 is created between distal end 33 of steerable catheter 30 and implant 60, i.e., proximal end 61 of implant 60 and proximal end 52 of proximal capsule portion 51. Gap 100 can range from 0-6 cm, e.g., 5 cm. As such, an axially-translatable region 102 of implant holder 40 is created at the portion of implant holder 40 which is exposed from distal end 33 of steerable catheter 30.

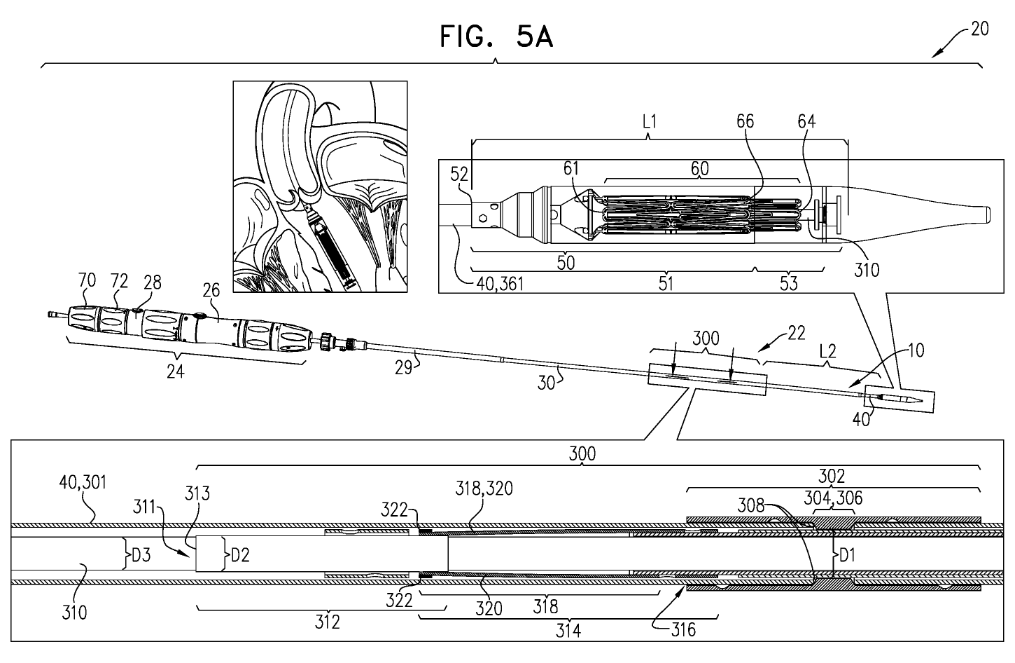

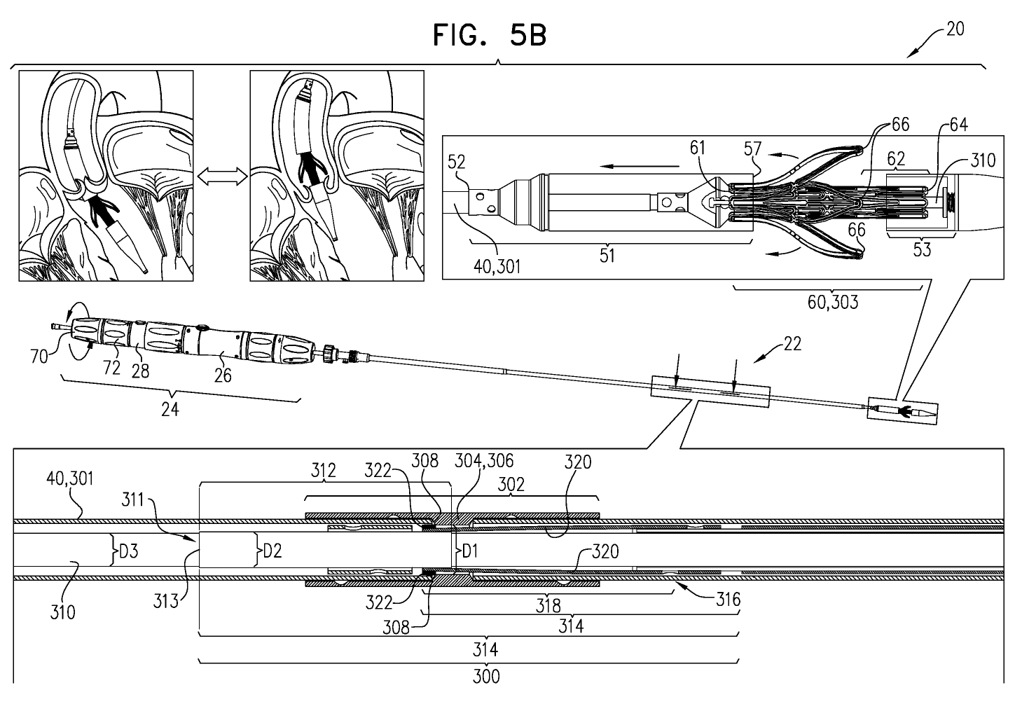

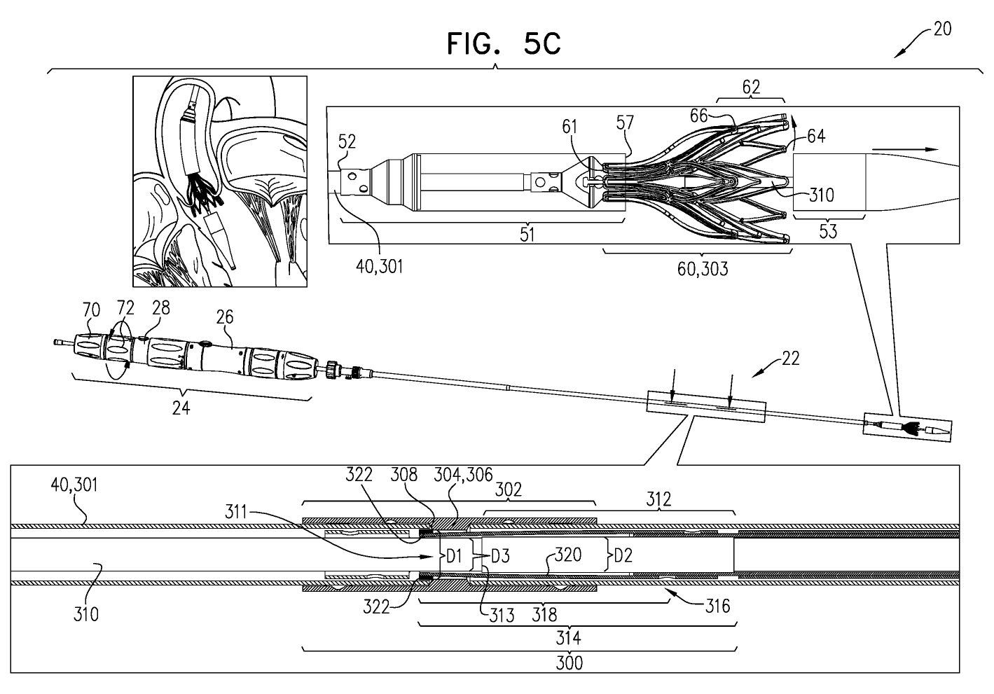

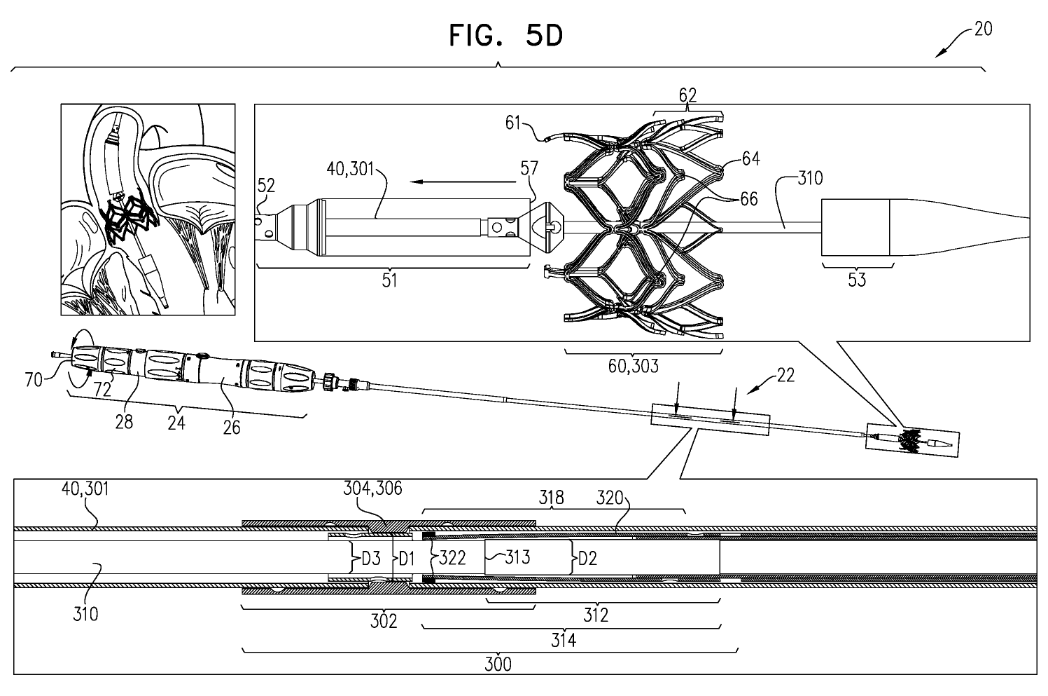

The ability of handle assembly 24 to transition between the fully-elongated state (FIG. 3A) and the fully-shortened state (FIG. 3B) provides for increased axial movement proximally and distally of axially-translatable region 102 of implant holder 40 during positioning of the prosthetic valve in the native valve. For example, for applications in which the prosthetic valve comprises a prosthetic aortic valve 303 (as shown in FIGS. 5A-D, the axially-translatable region of gap 100 enables the physician to properly position valve 303 while steerable catheter 30 remains stationary, and only implant holder 40 moves axially proximally and distally in order to achieve proper targeting of valve 303 at the native aortic valve.

As shown in FIG. 5B, prosthetic aortic valve 303 comprises one or more, e.g., 3-6, locators 66 which are configured to abut a downstream surface of the native aortic valve leaflets during positioning of prosthetic aortic valve 303. Locators 66 are radially-expandable from a compressed state within proximal capsule portion 51. Locators 66 provide feedback to enable the physician to properly position the prosthetic valve. In order to properly position valve 303, locators 66 can be advanced proximally and distally with respect to the native aortic valve leaflets. Axial movement of elongate-implant-holder-handle portion 28 distally within proximal receiving space 21 of steerable-catheter-handle portion 26 causes locators 66 to advance distally with respect to distal end 33 of steerable catheter 30, and vice versa. Movement of locators 66 (or any other portion of prosthetic valve 303) is performed while distal end 33 of steerable catheter 30 remains stationary in a position downstream of the native aortic valve, typically but not necessarily aligned with a longitudinal axis of the native aortic valve.

For some applications of the present invention, locators 66 may be radially expanded from their compressed state, within the left ventricle of the patient and then pulled and withdrawn proximally through the native aortic valve, as shown in FIG. 5B. In order to accomplish this proximal pulling of the locators 66, elongate-implant-holder-handle portion 28 moves proximally within proximal receiving space 21 of steerable-catheter-handle portion 26.

For some applications of the present invention, locators 66 may be radially expanded from their compressed state, within the aorta, downstream of the native aortic valve, as shown in FIG. 5B. Subsequently, locators 66 are advanced distally in order to abut the tips of locators 66 against the downstream surface of the native aortic valve leaflets. Locators 66 may be advanced distally by pushing distally tool 22, or by distally axially moving elongate-implant-holder-handle portion 28 with respect to steerable-catheter-handle portion 26.

Thus, such axial movement of elongate-implant-holder-handle portion 28 with respect to steerable-catheter-handle portion 26 enables fine axial adjustment of implant 60 during positioning and implantation of implant 60. This fine axial adjustment helps the physician properly target the positioning of the implant.

Additionally, the configuration of handle assembly 24 in the fully-shortened state (FIG. 3B) of handle assembly 24, creates increased navigability and trackability in region 102 of gap 100 and an overall narrower diameter of delivery tool 22 at region 102 during alignment, positioning, and unensheathing of implant 60 from within capsule 50. This narrowed diameter increases the navigability and trackability of region 102. For some applications in which implant 60 comprises prosthetic aortic valve 303, and region 102 is exposed during navigation through the aorta (e.g., in cases in which the aorta is difficult to navigate). For such applications, during advancement of implant 60 through the aorta, handle assembly is in the fully-shortened state (FIG. 3B) in order to enable region 102 of delivery tool 22 to a narrower diameter (i.e., the diameter of elongate implant holder 40) than when holder 40 is surrounded by steerable catheter 30. In such a case, the narrowed diameter increases navigability and trackability of region 102 during steering through the aorta.

As will be described hereinbelow, steerable-catheter-handle portion 26 of delivery tool 22 comprises (1) a first steerable-control-knob 76 for controlling lateral steering of a distal portion of steerable catheter 30, and (2) a second steerable-control-knob 78 disposed coaxially with respect to first steerable-control-knob 76. Second steerable-control-knob 78 controls flexible bending of the distal portion of steerable catheter 30. Second steerable-control-knob 78 facilitates axial movement of a flexing-control-screw 410, which is coupled to a flexing-control-pull wire 412 which facilitate flexing of the distal portion of steerable catheter 30. As shown in FIGS. 2 and 3A-B, for example, as the handle assembly transitions between the fully elongated state (FIG. 3A) and the fully shortened state (FIG. 3B), and prior to flexing of the distal portion of steerable catheter 30, screw 410 is disposed in a distal position. In order to effect flexing and bending of the distal portion of catheter 30, screw 410 is advanced proximally within the surrounding threading in order to apply tension to pull wire 412, as is described hereinbelow.

Reference is again made to FIG. 1. System 20 typically but not necessarily comprises a kit 14 which comprises delivery tool 22 and a packaging which houses delivery tool 22. When delivery tool 22 is disposed within the packaging, the distal portion 27 of the elongate-implant-holder-handle portion is disposed within the at least a proximal portion of proximal receiving space 21 of steerable-catheter-handle portion 26. It is to be noted that delivery tool 22 may be packaged within the housing in the fully elongated state of handle assembly 24, as shown in FIG. 3A, in the fully-shortened state of handle assembly 24, as shown in FIG. 3B, or in an intermediate state of handle assembly 24, as shown in FIG. 1.

In general, and in particular when delivery tool 22 is disposed within the packaging of kit 14, first thread 75 of elongate-implant-holder-handle portion 28 is matingly coupled to second thread 77 of steerable-catheter-handle portion 26.

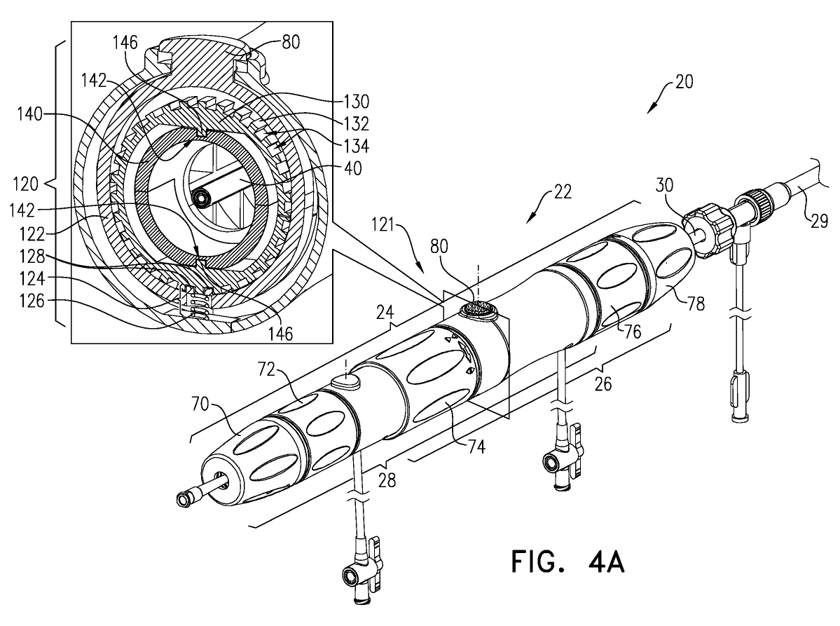

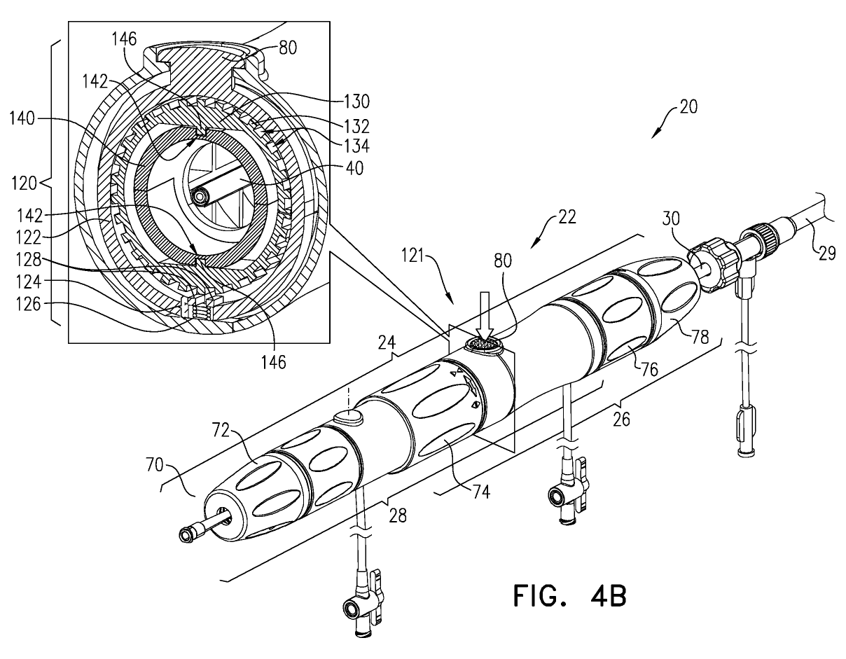

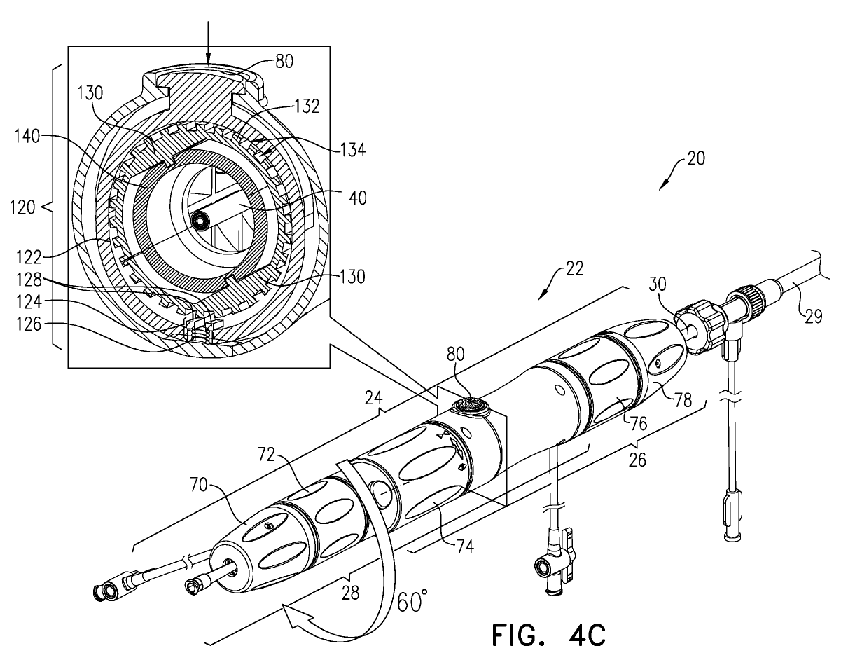

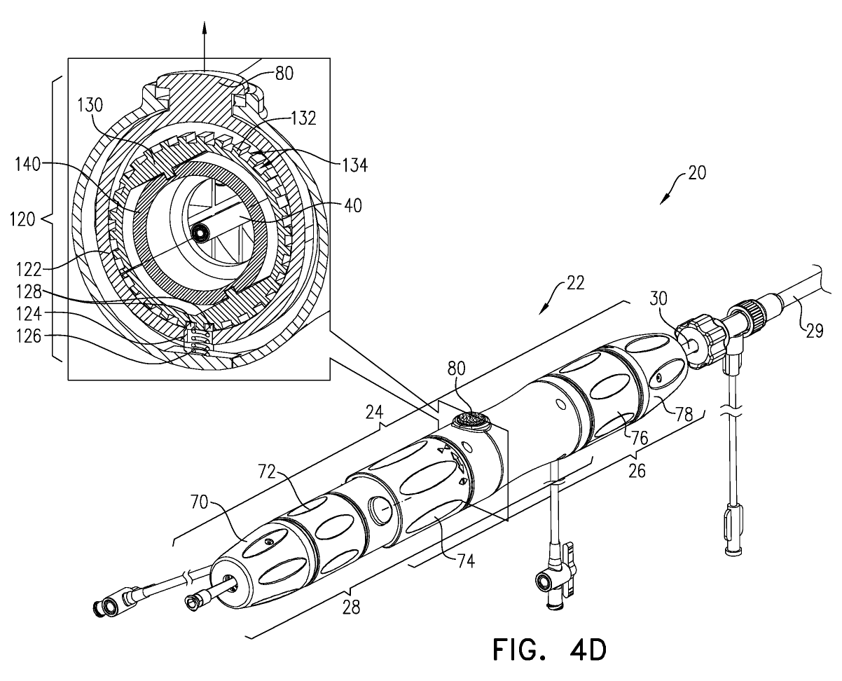

Reference is now made to FIGS. 4A-D, which are schematic illustrations of integrated handle assembly 24 of delivery tool 22 comprising a rotational locking mechanism 120 that controls rotational movement of (1) elongate-implant-holder-handle portion 28, elongate implant holder 40, and implant 60 with respect to (2) steerable-catheter-handle portion 26 and to the steerable catheter 30. Rotational locking mechanism 120 comprises a button 80 and a flexible ring 122 coupled to and actuatable by button 80 and comprises a spring-loaded engagement mechanism 124 that comprises a spring 126 at least one (e.g., two as shown) engagement tooth 128 coupled to spring 126.

Elongate-implant-holder-handle portion 28 comprises a distal adapter portion 121 which comprises a rotational adapter which facilitates rotation of (1) elongate-implant-holder-handle portion 28, elongate implant holder 40, and implant 60 with respect to (2) steerable-catheter-handle portion 26 and to steerable catheter 30. The rotational adapter comprises an adapter tube 140 shaped so as to define a first coupling 142 (e.g., a female coupling such as a groove, as shown). Steerable-catheter-handle portion 26 comprises a surrounding-ring 130 shaped so as to define a second coupling 146 (e.g., a male coupling, as shown) matingly coupled with first coupling 142 of adapter tube 140. Surrounding-ring 130 is shaped so as to define second coupling 146 at an inner surface of surrounding-ring 130, and a plurality of teeth 132 arranged circumferentially around an outer surface of surrounding-ring 130. Surrounding-ring 130 is shaped so as to define a plurality of spaces 134 alternatingly disposed with respect to the plurality of teeth 132.

Flexible ring 122 surrounds surrounding-ring 130 of steerable-catheter-handle portion 26. The diameter and shape of flexible ring 122 is controlled by button 80.

As shown in FIG. 4A, when button 80 is not pressed, flexible ring 122 is in a relaxed state and spring 126 is relaxed. In the relaxes states of ring 122 and spring 126, engagement teeth 128 are disposed within respective spaces 134 of surrounding-ring 130. In this state, locking mechanism 120 is in a locked state, and handle assembly 24 is in a rotationally locked state in which elongate-implant-holder-handle portion 28 is rotationally locked with respect to steerable-catheter-handle portion 26. In the rotationally locked state of handle assembly 24, axial movement of elongate-implant-holder-handle portion 28 with respect to steerable-catheter-handle portion 26 is permitted.

FIG. 4B shows handle assembly 24 in an unlocked state in which button 80 is pressed and in turn applies a pushing force to flexible ring 122. The pushing force applied to flexible ring 122 causes deformation of ring 122 such that ring 122 expands radially and a diameter of flexible ring 122 expands. Expanding of ring 122 applies a pushing force to spring 126 in order to disengage engagement teeth 128 from within the respective spaces 134 of surrounding-ring 130. In this state, locking mechanism 120 is in an unlocked state, and handle assembly 24 is in a rotationally unlocked state in which elongate-implant-holder-handle portion 28 is rotationally unlocked and is able to rotate with respect to steerable-catheter-handle portion 26, as shown in FIG. 4C. In the rotationally unlocked state of handle assembly 24, axial movement of elongate-implant-holder-handle portion 28 with respect to steerable-catheter-handle portion 26 is permitted.

Following rotation of elongate-implant-holder-handle portion 28, button 80 may be released from its pushed state in order to reengage locking mechanism 120 by releasing force on ring 122 such that spring 126 returns to its relaxed state as shown in FIG. 4D. In the relaxed state of spring 126, engagement teeth 128 are positioned within respective spaces 134 of surrounding-ring 130, and thereby, locking mechanism 120 returns to its locked state.

Reference is now made to FIGS. 5A-D, which are schematic illustrations of a safety mechanism 300 which controls unsheathing of implant 60 from within capsule 50, in accordance with some applications of the invention. Capsule 50 houses implant 60 in a radially compressed state during delivery, as shown in FIG. 5A. Elongate-implant-holder-handle portion 28 comprises knobs 70 and 72 for controlling the unsheathing of implant 60 from within capsule 50. Capsule 50 comprises a distal capsule portion 53 configured to house a distal portion of implant 60, and a proximal capsule portion 51 configured to house a proximal portion of implant 60. A proximal-capsule-knob 70 of handle portion 28 controls axial movement of proximal capsule portion 51, and a distal-capsule-knob 72 controls axial movement of distal capsule portion 53. Proximal and distal capsule portions 51 and 53 are axially movable with respect to each other.

It is to be noted that steerable catheter 30 is not shown in the enlarged images of FIGS. 5A-D for clarity of illustration, however, it is to be noted that steerable catheter 30 surrounds the components shown in the enlarged images of FIGS. 5A-D. Additionally, for some applications, a friction-reducing layer or coating (not shown for clarity of illustration) is disposed over stopper mechanism 300. The friction-reducing layer or coating is configured to reduce friction between the components of stopper mechanism 300 and steerable catheter 30. For some applications, the friction-reducing layer or coating may comprise fluorinated ethylene propylene (FEP), for example, or any other suitable material.

Additionally, prosthetic aortic valve 303 is shown without a fabric covering for clarity of illustration.

As described hereinabove elongate implant holder 40 is reversibly couplable to implant 60. Holder 40 comprises a proximal-capsule-moving-tube 301 that is coupled to proximal end 52 of proximal capsule portion 51. Proximal movement of proximal-capsule-moving-tube 301 moves proximal capsule portion 51 proximally, and distal movement of proximal-capsule-moving-tube 301 moves proximal capsule portion 51 distally.

Elongate implant holder 40 also comprises a distal-capsule-moving-tube 310 coupled to distal capsule portion 53 such that proximal movement of distal-capsule-moving-tube 310 moves distal capsule portion 53 proximally, and distal movement of distal-capsule-moving-tube 310 moves distal capsule portion 53.

Safety mechanism 300 comprises a proximal capsule locker 302 which surrounds and is coupled (e.g., typically, fixedly) to proximal-capsule-moving-tube 301. Proximal capsule locker 302 is shaped so as to define a midsection 304 shaped so as to define a midsection protrusion 306 which protrudes into a lumen of proximal-capsule-moving-tube 301. Midsection protrusion 306 has a midsection-inner-diameter D1 which is narrower than remaining portions of proximal capsule locker 302 such that midsection protrusion 306 of proximal capsule locker 302 forms a shoulder 308. Typically, midsection-inner-diameter D1 is 2.0 mm – 2.5 mm, e.g., 2.35 mm.

Distal-capsule-moving-tube 310 comprises a locking sleeve 312 that has a locking-sleeve-outer diameter D2, and a releasing portion 311 proximal to locking sleeve 312. Releasing portion 311 has a releasing-portion-outer diameter D3 that is narrower than locking-sleeve-outer diameter D2. For some applications, locking-sleeve-outer diameter D2 is 1.5 – 2.0 mm, e.g., 1.7 mm, and releasing-portion-outer diameter D3 is 1.0 – 1.5 mm, e.g., 1.34 mm.

Safety mechanism 300 comprises an intermediate tube 314 which surrounds and is coupled (e.g., typically, fixedly) to a portion of distal-capsule-moving-tube 310. Intermediate tube 314 has (1) an intermediate-tube lumen through which distal-capsule-moving-tube 310 and locking sleeve 312 slide, (2) a distal tubular portion 316, and (3) a collapsible proximal portion 318 formed of at least two elongate teeth 320 each having a proximal engaging element 322. Collapsible proximal portion 318 is maintained in a radially-pushed state in the presence of a structure which applies a radially-outward force to teeth 320, e.g., the presence of locking sleeve 312 between teeth 320, as shown in FIGS. 5A-B. Collapsible proximal portion 318 is disposed in a radially-collapsed state in the absence of the structure which applies the radially-outward force to teeth 320, e.g., the absence of locking sleeve 312 between teeth 320, as shown in FIGS. 5C-D.

FIGS. 5A-B show safety mechanism 300 in a locked state. In the locked state of safety mechanism 300, shoulder 308 of midsection protrusion 306 of proximal capsule locker 302 is disposed distally to proximal engaging elements 322 of intermediate tube 314.

FIG. 5A shows capsule portions 51 and 53 in a closed state. Proximal capsule locker 302 is disposed distally to teeth 320 and to proximal engaging elements 322 of intermediate tube 314.

FIG. 5B shows initial proximal movement of proximal capsule portion 51 in response to rotation of proximal-capsule-knob 70 of handle portion 28. Rotation of knob 70 withdraws proximally proximal-capsule-moving-tube 301 of elongate implant holder 40 which also withdraws proximally proximal capsule locker 302 such that proximal capsule locker 302 is disposed closer to teeth 320 and to proximal engaging elements 322 of intermediate tube 314. As such, shoulder 308 of midsection protrusion 306 of proximal capsule locker 302 is moved closer to the proximal engaging elements 322. In response to continued proximal movement of proximal capsule portion 51 and of proximal capsule locker 302 by proximal movement of proximal-capsule-moving-tube 301, shoulder 308 of the proximal capsule locker 302 abuts against proximal engaging elements 322.

In the locked state of safety mechanism 300, locking sleeve 312 of distal-capsule-moving-tube 310 is disposed within the lumen of intermediate tube 314 in a manner in which an outer surface of locking sleeve 312 of distal-capsule-moving-tube 310 is disposed between proximal engaging elements 322 and prevents collapsing of the at least two elongate teeth 320 such that proximal engaging elements 322 of teeth 320 remain engaged with shoulder 308 and remain in a state in which proximal engaging elements 322 restrict further proximal movement of proximal-capsule-moving-tube 301 and thereby of proximal capsule portion 51.

Reference is now made to FIGS. 1 and 5A, in prior to proximal movement of capsule portion 51 by distal-capsule-moving-tube 310 of elongate implant holder 40, a screw 71 which is controlled by knob 70 is disposed in a distal-most position. Rotation of knob 70 moves screw 71 proximally such that proximal capsule portion 51 is moved axially proximally.

In the locked state of safety mechanism 300, releasing portion 311 is disposed proximally to proximal engaging elements 322 and to shoulder 308 of midsection protrusion 306.

As shown in FIG. 5B, initial proximal retraction of proximal capsule portion 51 exposes the intermediate portion of prosthetic aortic valve 303 from within capsule portion 51 such that locators 66 expand radially. Such initial proximal retraction of proximal capsule portion 51 occurs following steering of steerable catheter 30 so as to position implant in the vicinity of the native aortic valve. For some applications, locators 66 are exposed within the left ventricle and then retracted proximally through the native aortic valve, e.g., by axial retraction of elongate-implant-holder-handle portion 28 as described hereinabove with reference to FIGS. 3A-B. Locators 66 are withdrawn to a position downstream of the native aortic valve leaflets. Subsequently, implant 60 is advanced distally such that locators 66 abut the downstream surface of the native aortic valve leaflets.

For some applications, locators 66 are exposed downstream of the native aortic valve leaflets and then pushed distally such that the tips of locators 66 abut against the downstream surface of the native aortic valve leaflets, e.g., by axial distal movement of elongate-implant-holder-handle portion 28 as described hereinabove with reference to FIGS. 3A-B. As described hereinabove with reference to FIGS. 4A-D, once locators 66 are exposed from within proximal capsule portion 51, elongate-implant-holder-handle portion 28 may be rotated with respect to steerable-catheter-handle portion 26 in order to properly position locators 66 with respect to the downstream surface of the leaflets of the native aortic valve.

At this stage of deployment, a distal end 64 of implant 60 remains compressed within distal capsule portion 53. Additionally, it is important that proximal end 61 of implant 60 remains compressed within proximal capsule portion 51. To this end, safety mechanism 300 ensures that proximal capsule portion 51 is not prematurely moved proximally so as to expose proximal end 61 of implant 60, which would result in premature release of implant 60 from delivery tool 22. That is, safety mechanism 300 maintains proximal capsule portion 51 in a position in which proximal capsule portion 51 surrounds and compresses proximal end 61 of implant 60 and thereby maintains coupling of implant 60 to delivery tool 22 until the operating physician determines that implant 60 is properly positioned with respect to the native tissue. In FIG. 5B, as distal end 57 of proximal capsule portion 51 is disposed distally to proximal end 61 of implant 60, thereby safety mechanism 300 retains the proximal portion of implant 60 compressed within proximal capsule portion 51.

Reference is now made to FIGS. 1 and 5B. Elongate-implant-holder-handle portion 28 of handle assembly 24 comprises an indicator 73, e.g., a button as shown, which is configured to pop out and make an audible sound to indicate to the operating physician when proximal capsule portion 51 reaches its pre-final-deployment position (i.e., the position of capsule portion 51 as shown in FIG. 5B, prior to its final movement, as shown in FIG. 5D in order to fully deploy prosthetic aortic valve 303). Indicator 73 provides an audible and visual indication to the physician to indicate deployment of locators 66, in addition to visualizing such deployment under imaging-based guidance.

Following the initial movement of proximal capsule portion 51 and the engaging of shoulder 308 with proximal engaging elements 322, distal-capsule-moving-tube 310 is moved axially distally through the intermediate-tube lumen of intermediate tube 314 so as to move distal capsule portion 53 distally, as shown in FIG. 5C. Distal-capsule-knob 72 is rotated in order to controls axial movement of distal capsule portion 53. Similarly to proximal-capsule-knob 70 as described hereinabove with reference to FIGS. 1 and 5A, distal-capsule-knob 72 also controls movement of a screw (not shown). Prior to movement of distal capsule portion 53, the screw controlled by knob 72 is disposed in a proximal-most position. Rotation of knob 72 moves the screw distally such that distal capsule portion 53 is moved axially distally. In response to the distal movement of distal capsule portion 53 by distal-capsule-moving-tube 310, (i) locking sleeve 312 moves distally beyond proximal engaging elements 322 of teeth 320 such that a proximal end 313 of locking sleeve 312 moves distally beyond proximal engaging elements 322, and (ii) releasing portion 311 of distal-capsule-moving-tube 310 moves within the lumen of intermediate tube 314 and in between (1) proximal engaging elements 322 and (2) at least a portion of the at least two elongate teeth 320. Such distal movement of distal-capsule-moving-tube 310 moves (i) the wider, locking-sleeve-outer diameter D2, distally beyond proximal engaging elements 322, and (ii) the narrower, releasing-portion-outer diameter D3 of releasing portion 311 between proximal engaging elements 322, which allows for the collapsing of the at least two elongate teeth 320 such that proximal engaging elements 322 of the teeth 320 are disengaged from shoulder 308 of midsection protrusion 306, and the further proximal movement of the proximal capsule is enabled (as is described hereinbelow with reference to FIG. 5D).

Distal movement of distal capsule portion 53 exposes a distal portion 62 and distal end 64 of implant 60 from within distal capsule portion 53 and enables distal portion 62 to radially expand. For applications in which implant 60 comprises prosthetic aortic valve, distal portion 62 comprises a distal, upstream skirt which is placed upstream of the native aortic valve and prevents paravalvular leaks. FIG. 5C shows distal movement of distal-capsule-moving-tube 310 axially through the lumen of intermediate tube 314 and thereby moves distal capsule portion 53 distally. As such, the distal, upstream skirt is exposed from within distal capsule portion 53 and sandwiches the native aortic valve leaflets between (1) the distal, upstream skirt and (2) the one or more radially-expandable locators 66.

FIGS. 5C-D represent safety mechanism 300 in an unlocked state.

Now that safety mechanism 300 is in the unlocked state, the proximal portion of implant 60 can be unsheathed from within proximal capsule portion 51 such that implant 60 is decoupled from tool 22. FIG. 5D shows additional rotation of proximal-capsule-knob 70 of handle portion 28 in order to further proximally withdraw proximal capsule portion 51. Proximal capsule portion 51 moves yet further proximally such that distal end 57 of proximal capsule portion 51 is disposed proximally to proximal end 61 of implant 60. The proximal portion of implant 60, i.e., the proximal-most end 61 of implant 60, is exposed from within proximal capsule portion, the proximal portion of implant 60 expands, and implant 60 is disengaged from delivery tool 22. For applications in which implant 60 comprises prosthetic aortic valve 303, the proximal, downstream portion of the valve 303 comprises a proximal, downstream skirt which is expanded downstream of the native aortic valve leaflets. Expanding of the proximal, downstream portion of the valve 303 further anchors valve 303 in the native aortic valve. In particular, expanding the proximal, downstream portion of the valve sandwiches the native aortic valve leaflets between (1) the distal, upstream skirt, and (2) locators 66 and the proximal, downstream skirt.

Following implantation of prosthetic aortic valve 303, delivery tool 22 is withdrawn proximally and extracted from within the body of the patient.

Reference is now made to FIGS. 5A-D. As shown in FIG. 5A, capsule 50 has a length L1 of 30 – 70 mm (e.g., 53 mm), which represents the stiff portion of delivery tool 22. A distal-most end of safety mechanism 300 is disposed at a distance L2 of 10 - 20 cm, e.g., 15 cm, from proximal end 52 of proximal capsule portion 51. It is further hypothesized by the inventors that positioning safety mechanism 300 at distal L2 from proximal end 52 of proximal capsule portion 51, increases the flexibility of the distal portion of catheter system 10 at the distal end which enables smoother steerability of catheter 30, and thereby of implant 60 at the distal portion of catheter system 10. It is important to increase flexibility of the distal portion of catheter system 10, in particular for embodiments in which catheter system 10 is advanced through the aorta. Therefore, positioning safety mechanism 300 at distal L2 from proximal end 52 of proximal capsule portion 51, enables the distal portion of catheter system 10 to navigate the aortic arch easier than if safety mechanism 300 were to be positioned at the distal portion of catheter system 10.

It is further hypothesized by the inventors that positioning safety mechanism 300 in the catheter section of tool 22 rather than in handle assembly 24, minimizes the components of handle assembly and allows for greater range of movements, i.e., more fine movements, of handle assembly which effects more focus on the mechanisms described herein relating to the steering of steerable catheter 30, the axial and rotational movement of elongate implant holder 40, and of the release of capsule portions 51 and 53. Additionally, positioning safety mechanism 300 toward the distal ends of elongate implant holder 40 and steerable catheter 30 creates less effective distance of forces being applied to capsule portions 51 and 53 than if they were to be applied from handle assembly 24.

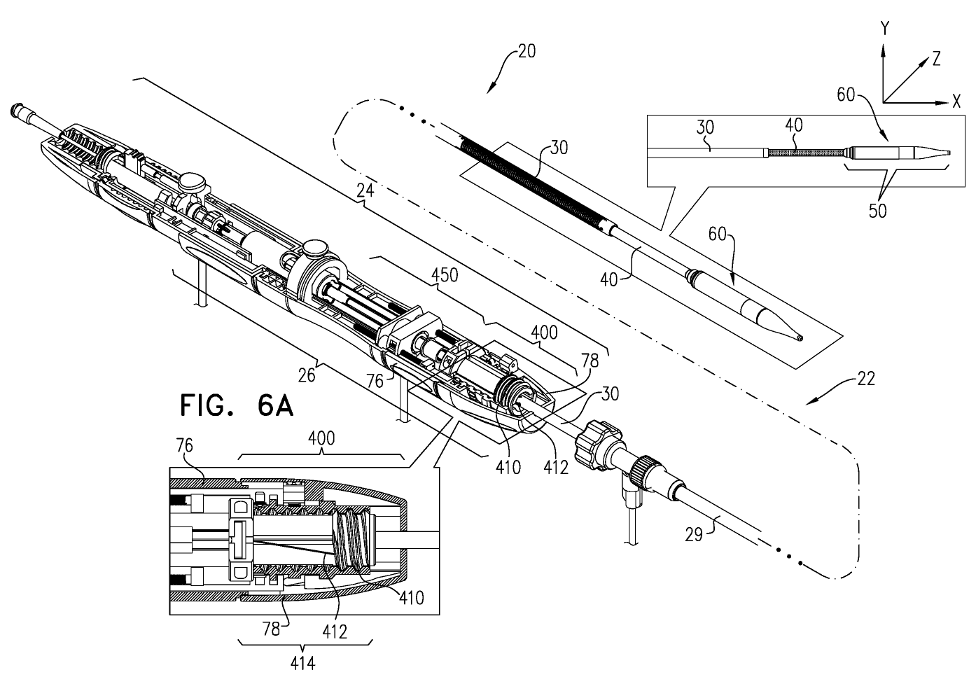

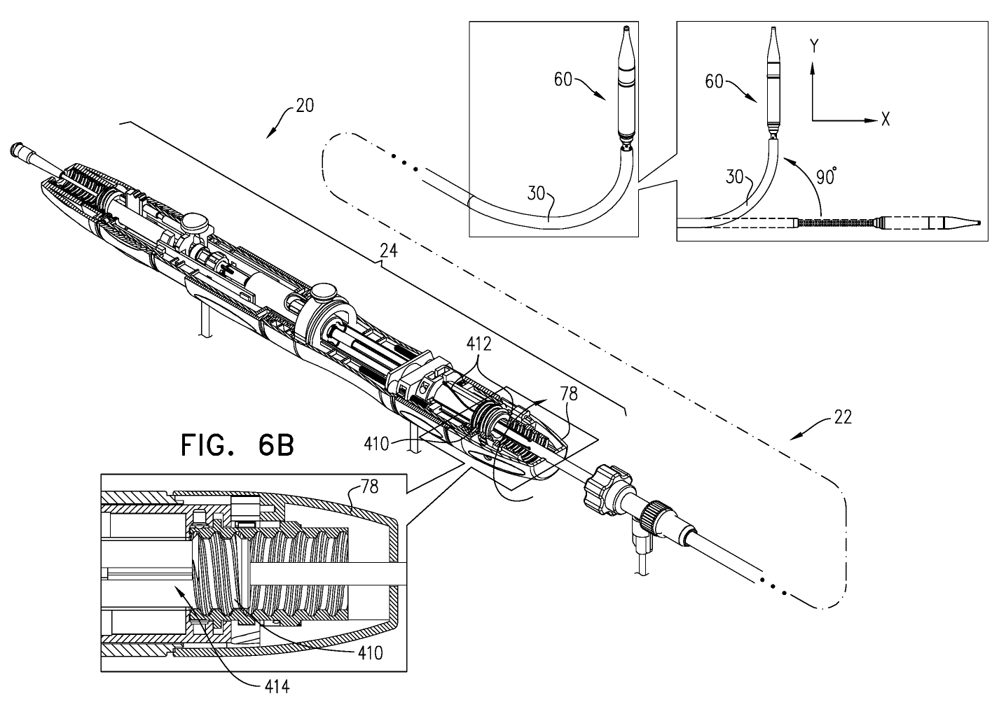

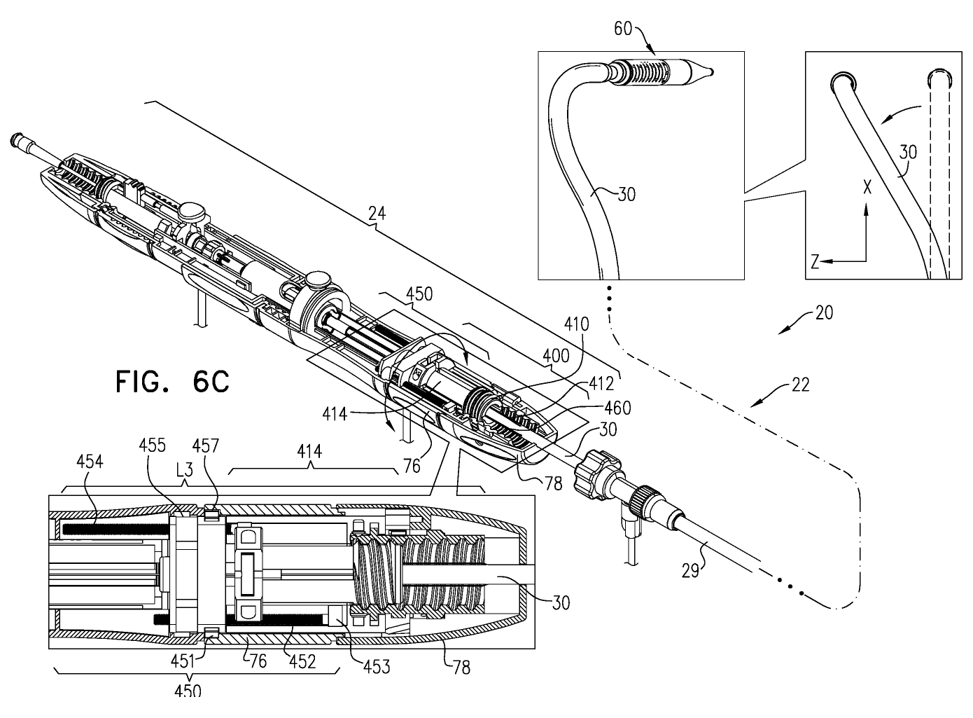

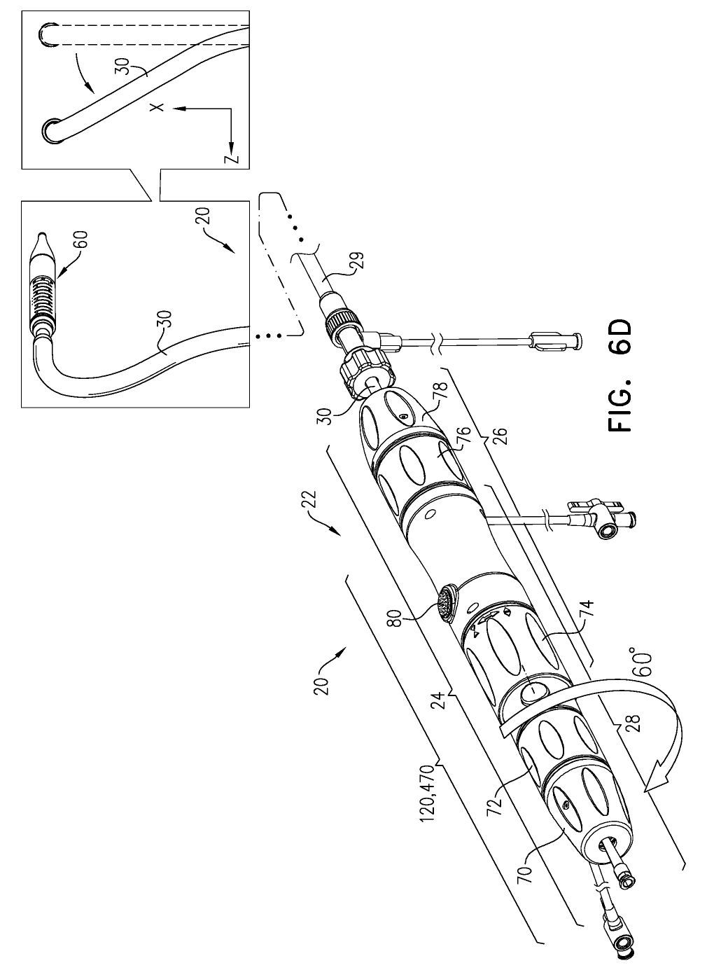

Reference is now made to FIGS. 6A-D, which are schematic illustrations of steerable assemblies 400, 450, and 470 for controlling steering of the distal tip portion of steerable catheter 30 and/or of the distal portion of elongate implant holder 40, in accordance with some applications of the invention. Steerable-catheter-handle portion 26 of handle assembly 24 comprises steerable assemblies 400 and 450. Steerable assembly 400 is configured to control flexible bending of the distal portion of steerable catheter 30, as shown in FIG. 6B. Steerable assembly 400 comprises flexing steerable-control-knob 78 which drives a flexing-control-screw 410. Flexing-control-screw 410 is coupled to a proximal portion of a flexing-control pull wire 412. A distal end of flexing-control pull wire 412 is coupled to the distal tip portion of steerable catheter 30. As shown in FIG. 6B, knob 78 is rotated which effects axial proximal movement of screw 410 within a flexing-control-screw-housing 414. Such axial proximal movement of screw 410 applies tension to flexing-control pull wire 412, and the distal tip portion of steerable catheter 30 is flexed and bent.

FIG. 6C shows steerable assembly 450 which is configured to control lateral steering of the distal portion of steerable catheter 30. Steerable assembly 450 comprises a flexing steerable-control-knob 76 which drives first and second gears 451 and 457 in opposite directions. Gear 451 is coupled to a first screw 452 such that rotation of gear 451 causes axial movement of first screw 452. Gear 457 is coupled to a second screw 454 such that rotation of gear 457 causes axial movement of second screw 454 in a direction opposite to first screw 452. Rotation of knob 76 causes equal and simultaneous rotation of first and second gears 451 and 457, such that first and second screws 452 and 454 simultaneously advance axially in opposite directions, as shown. First screw 452 is coupled to a first housing 453 which moves axially together with screw 452. Housing 453 couples a first-lateral-steering-pull wire 460 to screw 452. Second screw 454 is coupled to a second housing 455 which moves axially together with screw 454. Housing 455 couples a second-lateral-steering-pull wire (now shown) to screw 454.

As shown in FIG. 6C, knob 76 is rotated, which effects opposite movements of screws 452 and 454. Such axial movement of screws 452 and 454 applies and releases tension, respectively, to the first- and second-lateral-steering-pullwires in order to laterally steer the distal tip portion of steerable catheter 30. Such application and release of tension of the lateral-steering pullwires maximizes the ability of fine steering of the distal tip portion of steerable catheter 30.

As shown, screws 452 and 454 move axially at locations that are radially external to flexing-control-screw-housing 414. A proximal portion of flexing-control-screw-housing 414 is at times disposed between distal portions of screws 452 and 454 such that assemblies 400 and 450 axially overlap. Such positioning of screws 452 and 454 with respect to flexing-control-screw-housing 414 creates an integrated, axially-overlapping system of assemblies 400 and 450 which are concentric with respect to each other and thus, the overall combined length, L3 of assemblies 400 and 450 is shorter than if (1) flexing-control-screw-housing 414 and (2) screws 452 and 454 were to be fully coaxial (i.e., with no axial overlap). Typically, length L3 is 110.0 – 130.0 mm, e.g., 120.0 mm.

FIG. 6D shows steerable assembly 470 used in addition to or instead of steerable assembly 450. Steerable assembly 470 comprises all components of rotational locking mechanism 120 as described hereinabove with reference to FIGS. 4A-D. During the unlocked state of handle assembly 24, in which button 80 is pressed, elongate-implant-holder-handle portion 28 is rotationally unlocked and is able to rotate with respect to steerable-catheter-handle portion 26, as shown in FIG. 4C. During such rotation, torque builds up along elongate implant holder 40 such that the distal end of elongate implant holder 40 is steered laterally.

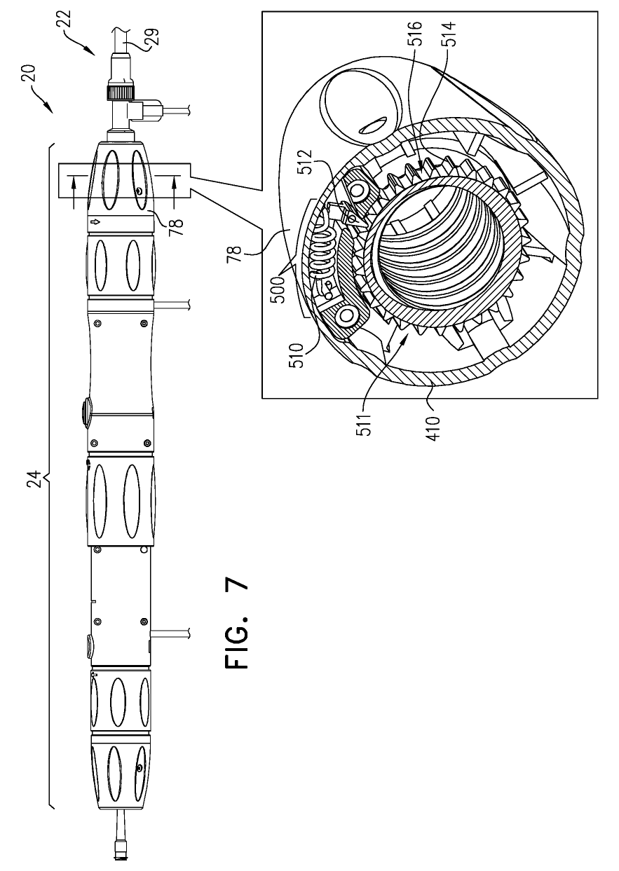

Reference is now made to FIG. 7, which is a schematic illustration of a clutch mechanism 500 which controls the amount of torque applied to pull wire 412, in accordance with some applications of the invention. Handle assembly 24 comprises clutch mechanism 500 integrated within steerable-control-knob 78 such that mechanism 500 is automatically activated after a number of rotations of knob 78 and too much tension is applied to pull wire 412. Pull wire 412 is operably coupled to knob 78 such that rotation of steerable-control-knob 78 applies tension to pull wire 412. Pull wire 412 is coupled to screw 410 such that rotation of steerable-control-knob 78 effects axial movement of screw 410 and affects the tension applied to pull wire 412. That is, proximal movement of screw 410 causes tension to be applied to pull wire 412, while distal movement of screw 410 causes tension to be released from pull wire 412. Clutch mechanism 500 comprises a spring 510 integrated within steerable-control-knob 78 and disposed circumferentially around a gear 511 coupled to flexing-control-screw 410. That is, spring 510 is aligned along an arc radially externally to screw 410. Spring 510 is coupled to a pin 512 which engages spaces 516 between teeth 514 of gear 511 when torque is applied to pull wire 412 under a given threshold. When pin 512 is engaged between teeth 514 of gear 511, rotation of knob 78 facilitates axial advancement of screw 410 in order to apply tension to pull wire 412 and effect steering of steerable catheter 30. Once the torque exceeds the threshold, spring 510 is under sufficient tension that spring 510 pulls on pin 512 in order to disengage pin 512 from within spaces 516, and continued rotation of knob 78 will not affect rotation of gear 511, and screw 410 will not advance axially such that additional tension and torque is not applied to pull wire 412. In such cases, steering of steerable catheter is not affected.

To aid the user in knowing when such a tension force is applied to these components, clutch mechanism 500 is used such that mechanism 500 releases when a certain torque is reached. It is advantageous for the user to know when clutch mechanism 500 is engaging. Therefore, as shown in FIG. 7, clutch mechanism 500 comprises a ratchet clutch, whereby the operator may be notified that the clutch is engaged due to the clutch producing audible clicks.

Reference is now made to FIGS. 1-7. It is to be noted that although the prosthetic valve described in are described as being configured for implantation at a native aortic valve of the patient, the prosthetic valve may also be configured, mutatis mutandis, to be implanted at other cardiac valves of the patient, e.g., the pulmonary valve, the mitral valve and the tricuspid valve.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A delivery tool for delivering an implant to a heart of a patient, the delivery tool comprising:
a delivery catheter having a lumen therethrough;
a distal capsule portion configured to house a distal portion of the implant;
a proximal capsule portion configured to house a proximal portion of the implant, the proximal and distal capsule portions being movable with respect to each other;
an elongate implant holder slidable within the lumen of the delivery catheter and reversibly couplable to the implant, the elongate implant holder comprising:
a proximal-capsule-moving-tube coupled to a proximal end of the proximal capsule portion such that proximal movement of the proximal-capsule-moving-tube moves the proximal capsule portion proximally, and distal movement of the proximal-capsule-moving-tube moves the proximal capsule portion distally;
a distal-capsule-moving-tube coupled to the distal capsule portion such that proximal movement of the distal-capsule-moving-tube moves the distal capsule portion proximally, and distal movement of the distal-capsule-moving-tube moves the distal capsule portion distally; and
a safety mechanism comprising:
a proximal capsule locker which surrounds and is coupled to the proximal-capsule-moving-tube, the proximal capsule locker being shaped so as to define a midsection protrusion which protrudes into a lumen of the proximal-capsule-moving-tube, the midsection having a midsection-inner-diameter which is narrower than remaining portions of the proximal capsule locker such that the midsection of the proximal capsule locker forms a shoulder;
a locking sleeve coupled to and surrounding the distal-capsule-moving-tube, the locking sleeve having a locking-sleeve-outer diameter, wherein the distal-capsule-moving-tube has a releasing portion proximal to the locking sleeve, the releasing portion having a releasing-portion-outer diameter that is narrower than the locking-sleeve-outer diameter, and
an intermediate tube which surrounds the distal-capsule-moving-tube, the intermediate tube having:
an intermediate-tube lumen through which (i) the distal-capsule-moving-tube, and (ii) and the locking sleeve slide,
a distal tubular portion, and
a collapsible proximal portion formed of at least two elongate teeth each having a proximal engaging element,
wherein:
in a locked state of the safety mechanism:
the shoulder of the proximal capsule locker is disposed distally to the proximal engaging elements of the intermediate tube,
in response to proximal movement of the proximal capsule portion and of the proximal capsule locker by the proximal-capsule-moving-tube, the shoulder of the proximal capsule locker is moved closer to the proximal engaging elements,
in response to continued proximal movement of the proximal capsule portion and of the proximal capsule locker by the proximal-capsule-moving-tube, the shoulder of the proximal capsule locker abuts against the proximal engaging elements, and
the locking sleeve of the distal-capsule-moving-tube is disposed within the lumen of the intermediate tube and in a manner in which an outer surface of the locking sleeve of the distal-capsule-moving-tube is disposed between the proximal engaging elements and prevents collapsing of the at least two elongate teeth such that the proximal engaging elements of the teeth remain engaged with the shoulder of the proximal capsule locker and remain in a state in which the proximal engaging elements restrict further proximal movement of the proximal capsule portion,
following movement of the proximal capsule portion and engaging of the shoulder with the proximal engaging elements, the distal-capsule-moving-tube is configured to be moved axially distally through the intermediate-tube lumen so as to move the distal capsule portion distally and transition the safety mechanism into an unlocked state, and
in response to distal movement of the distal capsule portion by the distal-capsule-moving-tube, the locking sleeve moves distally beyond the proximal engaging elements of the teeth and the releasing portion of the distal-capsule-moving-tube moves within the lumen of the intermediate tube and in between (1) the proximal engaging elements and (2) at least a portion of the at least two elongate teeth in a manner which allows for the collapsing of the at least two elongate teeth such that the proximal engaging elements of the teeth are disengaged from the shoulder, and the further proximal movement of the proximal capsule portion is enabled.

2. The delivery tool according to claim 1, wherein the delivery catheter comprises a steerable catheter.

3. The delivery tool according to claim 1, further comprising:
a proximal-capsule-knob configured to control axial movement of the proximal capsule portion; and
a distal-capsule-knob configured to control axial movement of the distal capsule portion.

4. The delivery tool according to claim 1, further comprising:
a first steerable-control-knob for controlling lateral steering of a distal portion of the steerable catheter, and
a second steerable-control-knob disposed coaxially with respect to the first steerable-control-knob, the second steerable-control-knob being configured for controlling flexible bending of the distal portion of the steerable catheter.

5. The delivery tool according to claim 1, further comprising at least one of the following features:
(a) the midsection-inner-diameter is 2.0mm – 2.5mm;
(b) the locking-sleeve-outer diameter is 1.5 – 2.0mm; and
(c) the releasing-portion-outer diameter D3 is 1.0 – 1.5mm.

6. The delivery tool according to claim 1, wherein the native heart valve includes a native aortic valve, and wherein the implant comprises:
a proximal, downstream skirt which is configured to be expanded downstream of native aortic valve leaflets;
one or more radially-expandable locators configured to abut a downstream surface of the native aortic valve leaflets during the positioning of the implant within the native heart valve; and
a distal, upstream skirt which is configured to be placed upstream of the native aortic valve and sandwich the native aortic valve leaflets between (1) the distal, upstream skirt and (2) the one or more radially-expandable locators and the proximal, downstream skirt.

7. The delivery tool according to claim 6, wherein the one or more radially-expandable locators are expandable at a position downstream of the native aortic valve leaflets in response to the proximal movement of the proximal capsule portion of the proximal capsule locker by the proximal movement of the proximal-capsule-moving-tube.

8. The delivery tool according to claim 6, wherein the one or more radially-expandable locators are expandable within a left ventricle of the heart of the patient.

9. The delivery tool according to claim 6, wherein the distal, upstream skirt is configured to:
be exposed from within the distal capsule portion during the distal movement of the distal-capsule-moving-tube axially through the intermediate-tube lumen, and to the movement of the distal capsule portion distally, and
sandwich the native aortic valve leaflets between (1) the distal, upstream skirt and (2) the one or more radially-expandable locators.

10. The delivery tool according to claim 9, the proximal capsule portion is further moveable proximally by further movement of the proximal-capsule-moving-tube such that:
a proximal end of the implant is exposed from within the proximal capsule portion,
the proximal, downstream skirt is configured to expand, and
the native aortic valve leaflets are sandwiched between (1) the distal, upstream skirt and (2) the one or more radially-expandable locators and the proximal, downstream skirt.

11. A method for delivering an implant to a heart of a patient using a delivery tool, the delivery tool comprising:
a delivery catheter having a lumen therethrough;
a distal capsule portion configured to house a distal portion of the implant;
a proximal capsule portion configured to house a proximal portion of the implant, the proximal and distal capsule portions being movable with respect to each other;
an elongate implant holder slidable within the lumen of the delivery catheter and reversibly couplable to the implant, the elongate implant holder comprising:
a proximal-capsule-moving-tube coupled to a proximal end of the proximal capsule portion such that proximal movement of the proximal-capsule-moving-tube moves the proximal capsule portion proximally, and distal movement of the proximal-capsule-moving-tube moves the proximal capsule portion distally;
a distal-capsule-moving-tube coupled to the distal capsule portion such that proximal movement of the distal-capsule-moving-tube moves the distal capsule portion proximally, and distal movement of the distal-capsule-moving-tube moves the distal capsule portion distally; and
a safety mechanism comprising:
a proximal capsule locker which surrounds and is coupled to the proximal-capsule-moving-tube, the proximal capsule locker being shaped so as to define a midsection protrusion which protrudes into a lumen of the proximal-capsule-moving-tube, the midsection having a midsection-inner-diameter which is narrower than remaining portions of the proximal capsule locker such that the midsection of the proximal capsule locker forms a shoulder;
a locking sleeve coupled to and surrounding the distal-capsule-moving-tube, the locking sleeve having a locking-sleeve-outer diameter, wherein the distal-capsule-moving-tube has a releasing portion proximal to the locking sleeve, the releasing portion having a releasing-portion-outer diameter that is narrower than the locking-sleeve-outer diameter,
an intermediate tube which surrounds the distal-capsule-moving-tube, the intermediate tube having:
an intermediate-tube lumen through which (i) the distal-capsule-moving-tube, and (ii) and the locking sleeve slide,
a distal tubular portion, and
a collapsible proximal portion formed of at least two elongate teeth each having a proximal engaging element,
advancing the delivery catheter through vasculature of the patient;
positioning the implant within a native heart valve of the heart, wherein in the step of advancing and the step of positioning the safety mechanism is in a locked state in which the shoulder of the proximal capsule locker is disposed distally to the proximal engaging elements of the intermediate tube;
proximally moving the proximal capsule portion of the proximal capsule locker by proximally moving the proximal-capsule-moving-tube, such that responsively to the proximally moving of the proximal-capsule-moving-tube, moving the shoulder of the proximal capsule locker proximally and closer to the proximal engaging elements;
further proximally moving the proximal capsule portion and the proximal capsule locker by further proximally moving the proximal-capsule-moving-tube, such that in response to the further proximally moving of the proximal-capsule-moving-tube, abutting the shoulder of the proximal capsule locker against the proximal engaging elements, and
wherein, in the locked state of the safety mechanism, the locking sleeve of the distal-capsule-moving-tube is disposed within the lumen of the intermediate tube and in a manner in which an outer surface of the locking sleeve of the distal-capsule-moving-tube is disposed between the proximal engaging elements and prevents collapsing of the at least two elongate teeth such that the proximal engaging elements of the teeth remain engaged with the shoulder of the proximal capsule locker and remain in a state in which the proximal engaging elements restrict further proximal movement of the proximal capsule portion,
following the step of further proximally moving the proximal-capsule-moving-tube, transitioning the safety mechanism to an unlocked state by distally moving the distal-capsule-moving-tube axially through the intermediate-tube lumen, and thereby moving the distal capsule portion distally;
responsively to the moving the distal capsule portion distally by the distal-capsule-moving-tube, moving the locking sleeve distally beyond the proximal engaging elements of the teeth, and moving the releasing portion of the distal-capsule-moving-tube within the lumen of the intermediate tube and in between (1) the proximal engaging elements and (2) at least a portion of the at least two elongate teeth in a manner which allows for the collapsing of the at least two elongate teeth such that the proximal engaging elements of the teeth are disengaged from the shoulder; and
yet further proximally moving the proximal capsule portion by yet further proximally moving the proximal-capsule-moving-tube.

12. The method according to claim 11, wherein the delivery catheter comprises a steerable catheter.

13. The method according to claim 11, wherein the delivery tool further comprises:
a proximal-capsule-knob configured to control axial movement of the proximal capsule portion; and
a distal-capsule-knob configured to control axial movement of the distal capsule portion.

14. The method according to claim 11, wherein the delivery tool further comprises:
a first steerable-control-knob for controlling lateral steering of a distal portion of the steerable catheter, and
a second steerable-control-knob disposed coaxially with respect to the first steerable-control-knob, the second steerable-control-knob being configured for controlling flexible bending of the distal portion of the steerable catheter.

15. The method according to claim 11, further comprising at least one of the following features:
(a) the midsection-inner-diameter is 2.0mm – 2.5mm;
(b) the locking-sleeve-outer diameter is 1.5 – 2.0mm; and
(c) the releasing-portion-outer diameter D3 is 1.0 – 1.5mm.

16. The method according to claim 11, wherein the native heart valve includes a native aortic valve, and wherein the implant comprises:
a proximal, downstream skirt which is configured to be expanded downstream of native aortic valve leaflets;
one or more radially-expandable locators configured to abut a downstream surface of the native aortic valve leaflets during the positioning of the implant within the native heart valve; and
a distal, upstream skirt which is configured to be placed upstream of the native aortic valve and sandwich the native aortic valve leaflets between (1) the distal, upstream skirt and (2) the one or more radially-expandable locators and the proximal, downstream skirt.

17. The method according to claim 16, wherein proximally moving the proximal capsule portion of the proximal capsule locker by proximally moving the proximal-capsule-moving-tube comprises expanding the one or more radially-expandable locators at a position downstream of the native aortic valve leaflets.

18. The method according to claim 16, wherein the method further comprises advancing the proximal capsule portion within a left ventricle prior to positioning the implant within the native heart valve;
wherein proximally moving the proximal capsule portion of the proximal capsule locker by proximally moving the proximal-capsule-moving-tube comprises
expanding the one or more radially-expandable locators within the left ventricle, and wherein the method further comprises:
proximally withdrawing the one or more radially-expandable locators to a position downstream of the native aortic valve leaflets; and
distally advancing the implant such that the one or more radially-expandable locators abut the downstream surface of the native aortic valve leaflets.

19. The method according to claim 16, wherein proximally moving the proximal capsule portion of the proximal capsule locker by proximally moving the proximal-capsule-moving-tube comprises exposing the one or more radially-expandable locators from within the proximal capsule portion and expanding the one or more radially-expandable locators, and wherein the method further comprises distally advancing the implant such that the one or more radially-expandable locators abut the downstream surface of the native aortic valve leaflets.

20. The method according to claim 16, wherein distally moving the distal-capsule-moving-tube axially through the intermediate-tube lumen, and thereby moving the distal capsule portion distally, comprises exposing the distal, upstream skirt from within the distal capsule portion and sandwiching the native aortic valve leaflets between (1) the distal, upstream skirt and (2) the one or more radially-expandable locators;
wherein yet further proximally moving the proximal capsule portion by yet further proximally moving the proximal-capsule-moving-tube comprises exposing a proximal end of the implant from within the proximal capsule portion and sandwiching the native aortic valve leaflets between (1) the distal, upstream skirt and (2) the one or more radially-expandable locators and the proximal, downstream skirt; and
wherein exposing the proximal end of the implant from within the proximal capsule portion comprises disengaging the implant from the delivery tool.
